(19) United States
(12) Patent Application Publication
(10) Pub. No.: US 2020/0058381 A1
(43) Pub. Date: Feb. 20, 2020

(54) System and Method for Auditing, Monitoring, Recording, and Executing Healthcare Transactions, Communications, and Decisions

(71) Applicant: Miteshkumar Ishwarbhai Patel, San Leandro, CA (US)

(72) Inventor: Miteshkumar Ishwarbhai Patel, San Leandro, CA (US)

(21) Appl. No.: 16/102,892

(22) Filed: Aug. 14, 2018

(51) Int. Cl. G16H 10/60; G06Q 40/08

(52) U.S. Cl. CPC G16H 10/60; G06Q 40/08

(57) ABSTRACT

Systems and methods are disclosed for monitoring, recording, verifying, auditing, and executing healthcare communications, decisions, and transactions using centralized storage and blockchain technology. The healthcare communications relate to communications between a patient, doctor, specialist, healthcare provider, a private or government entity that processes and reimburses healthcare providers, insurance provider, and a healthcare facility.

Systems and methods are disclosed for processing, monitoring, recording, verifying, auditing, and executing healthcare communications, decisions, and transactions using centralized storage and blockchain technology. The healthcare communications relate to communications between a patient, doctor, specialist, healthcare provider, a private or government entity that processes and reimburses healthcare providers, insurance provider, and a healthcare facility.

In one embodiment, the system processes a healthcare transaction that relates to medical care provided for a patient that has a medical health plan. The method involves providing a centralized control manager for obtaining, storing, and analyzing healthcare transaction data.

The centralized control manager is connected to a central database and utilizes the central database for storing healthcare data. The centralized control manager operates through a processor. The processor executes instructions stored in the database leading to the centralized control manager performing its functions.

The centralized control manager is electronically and communicatively connected to at least two entities that are part of the authorized entities within the computer network. These entities may include a patient, doctor, primary physician, specialist, payer system, health plan provider, and government entity such as Medicare. The centralized control manager allows bidirectional communication with the centralized control manager and other authorized entities that are allowed to communicate with the centralized control manager and use an electronic device over a computerized network. Communications directly between the entities is also contemplated.

The entities electronically execute a healthcare transaction. The centralized control manager records each executed healthcare transaction into the centralized database as well as posts the transaction to the blockchain. Each entity performing the transaction also posts the details of the transaction to the blockchain. Details include the time and date of transaction, the parties involved, and other details of medical care provided to the patient relating to the transaction.

A prediction engine, utilizing machine learning technology, uses the data of all the transactions obtained to continuously enhance and predict the outcome of future transactions and make suggestions as needed. If any transaction deviates above a certain limit, e.g. a medical claim is filed or a medical code is entered that does not fall within a set limit, the prediction engine flags the transaction for review and optionally alerts the entities involved in the transaction.

All of the transactions are replicated to the blockchain by multiple entities in the computer network. These include the transacting entity, the receiving entity, and the centralized control manager. Posting to the blockchain allows for verification and authentication of the transactions.

System and methods also allow automated claim adjudication and dispute resolution between two parties in the healthcare environment. The claim adjudication and dispute resolution relate to medical care provided for patients who have a medical health plan.

The system includes a server computer and a computing device. The server computer is comprised of a central database and a processor as well as a server application that is stored in the central database. The computing device includes an electronic device having a client processor and client database. Both the server and client are communicatively connected.

In this embodiment, a Healthcare Provider executes a transaction that is recorded in the centralized database as well as in the blockchain. The server computer electronically stores the data of the healthcare transaction in the central database and provides access of the transaction to the Healthcare Provider, Payer, and other relevant parties.

Thereafter, the Healthcare Provider files a dispute with the Payer System. The server computer electronically sends an alert to the relevant and authorized parties informing them that a dispute has been filed. The server processor then obtains data from the central database, blockchain, and any other relevant data provided by other parties in the computer network that relate to the disputed healthcare transaction. The processor then parses the data, resolves the dispute, and posts the results to the centralized server and blockchain.

### BACKGROUND OF THE INVENTION

### Field of the Invention

This application is a set of systems and methods for auditing, monitoring, recording, verifying, and executing healthcare communications, decisions, and transactions using centralized storage and blockchain technology. More specifically, the system serves as a communication tool between patients, healthcare providers, healthcare related government entities, insurance carriers, and healthcare facilities. The application also relates to recording and executing of decisions and transactions between the parties such that healthcare processing is performed through a centralized blockchain system.

### Description of the Related Art

The United States Healthcare industry has several challenges. At a minimum, these difficulties include delays in processing, delays in communication, mismanagement of healthcare data, issues with timely approvals or denials for treatments or specialist visits, lack of understanding of health care plan coverage, and incorrect or delayed billing. Another challenge exists due to the numerous systems that operate differently. Complex rules for processing transactions. Different rules for different types of insurances.

These problems and delays are rarely reported or corrected in a timely manner and can result in auditors uncovering major problems often too late causing huge penalties, and in some circumstances, closure to the organization. This leads to patient receiving substandard, delayed, or denied care. In a typical patient—doctor relationship, there is an element of trust and patients feel uncomfortable in reporting issues such as lack of care, delayed referrals, and other unmet health care need experienced with their doctor visit.

Delays, errors, and improper handling are prevalent in a healthcare plan provider, healthcare facility, and government entity setting where each have to interact with the other for processing healthcare data, healthcare approval, and moving patient care forward. It is not humanly possible to monitor every transaction to prevent abuse or errors. Much too often, providers are too busy caring for their patients to worry about filing appeals and complaints. In some cases, the patients are too sick to file complaints.

Most healthcare plans and providers require patients to undergo preventative care visits and preventative physical and lab examinations. A large percentage of the population fails to receive these recommended preventive services and several do not receive the full range of clinically indicated services for acute and chronic conditions. Many reasons including delayed patient authorizations, improper communication between healthcare providers and facilities, improper billing add to the problem causing patients to give up or not go for their preventative care visit. Sometimes the providers receive a payment based on capitation per month for all of their patients so they do not care if the patient comes to the office or not and they will still be paid. Patients end up in the ER or Urgent Care when providers are not available resulting in increased healthcare expenses.

In yet another example, patient care requires specialists, additional screening, lab work, or advanced care than may require multiple doctor visits or visits to a specialist or for a special medical procedure. In such a setting, a patient's health insurer or plan provided by the health care service needs to approve the care prior to it is incurred. This is sometimes called prior authorization, prior approval or precertification which provides some level of assurance that the advanced care, either partially or in full may be covered by the health care plan. Even then so, preauthorization isn't a promise your health insurance or plan will cover the cost, it just means they authorize the treatment and the costs may have to be shared by the patient. The messaging between the entities, the delays in the approvals, the data provided, is often confusing. Further, lack of accountability between the institutions leave a patient with a high cost to cover on their own or cause the patient to give up pursuing advanced care in many instances. These delays can be determinantal and in some cases be life threatening. Many United States Veterans that had delays in approvals and access to higher care died because of lack of care to their critical medical conditions. A two-month delay in a visit to an oncologist can mean the different between stage 2 Cancer or Stage 4 cancer. To put it in perspective, it is the difference between life and death.

In many instances, such as with Medicare or State Sponsored Medical Assistance Programs, like Medi-cal in California, the local State or Federal Government pays for the health care plan provider and the healthcare facility for administering care to the patients that are part of the State or Federal sponsored plan.

In such a government sponsored setting, the healthcare plan and the healthcare provider, such as a clinic, medical facility, Doctor's office, hospital, Urgent Care facility submit a bill to the government for payment of the services rendered to the patient. This is known as Medicare reimbursement, or Medi-cal or other state/federal program named reimbursement. This refers to the payments that hospitals and providers receive in return for services rendered to Medicare beneficiaries. The reimbursement prices for these services are set by the government entity, such as Medicare, and are typically less than the amount billed or the amount that a private insurance company would pay. In doing so, the government tries to have a consistency across healthcare facilities and ensure that particular treatment is not overcharged.

In the United States, healthcare providers bill for medical services using CPT codes. These codes represent medical services provided to a patient. ICD codes, also known as diagnosis codes, are also used to support the necessity of the medical services being rendered. The healthcare providers are reimbursed based on the complexity of the procedure and the complexity of the patient's diagnosis. To obtain reimbursement from the government, these healthcare providers code the patient care with all the codes associated with the treatment and submit them for reimbursement and payment. The payments vary based on where the service was provided, what the diagnosis was, the type of provider visited, and if the service was a result of another service or commonly performed with another service. There are also other rules which may impact payments.

There are several reported cases of fraud and improper or excessive coding by healthcare facilities. Medicare fraud, which is the claiming of Medicare healthcare reimbursement to which the claimant is not entitled, has become a big issue as many try and collect money from the government program illegitimately. Some healthcare facilities take advantage of the lack of monitoring by Medicare and lack of transparency into their practices by fraudulently claiming a higher reimbursement. Sometimes a patient living in Florida may be receiving service fraudulently in California. A patient with a cataract operation in right eye may get another operation of the same eye and because the systems do not talk to one another, it may get paid again.

Long term hospitalization of patients who do not need long term care remains an example of such fraud. Medicare typically pays for the entire stay when the stay is 30-days or less. If the patient's stay at a healthcare facility exceeds 30-days, Medicare will only pay a portion of the stay leaving the remaining balance as the patient's responsibility. Healthcare facilities have been reported to abuse this 30-day rule in an attempt to recoup the maximum allowable Medicare payment by unnecessarily keeping patients hospitalized for longer terms, i.e., closer to the 30-day threshold even when they do not need such long-term care.

As such, a better healthcare system that provides transparency, accountability, and reduces errors and fraud, and expedites care authorizations while keeping the required parties in the loop is desperately needed. Some groups have been caught denying and delaying care or denying and delaying payments and then modifying database transactions to state that proper notifications have been done on a timely basis. In reality, they never sent of a letter but the add data into the system that a letter was sent on this date time. Some groups have said they have sent the payment but the checks were never mailed out. Other groups claim they never received an authorization or claims but provider has submitted them regularly. Some groups modify database transactions and change dates to avoid untimely processing interest penalties. To track this is humanly impossible unless an auditor sits full time at the site and in some cases multiple auditors may be required. In fact, most entities are audited once every few years and when an auditor does go onsite, they choose 20-30 transactions to audit out of hundreds of thousands. For the entity the time it takes to prepare for an audit is enormous and sometimes they discover faults which they did not pick up earlier causing the entity to try to fudge the data to pass the audit. The auditor is also too busy verifying data which is hard to gather and time consuming to verify. They really have no way to verify if a letter was actually mailed out or someone just entered in the system that a letter was sent. Sometimes the auditors change, people who handle patient complaints change so there is no central way of tracking where there is a growing concern. Often a true problem which may have impacted patients negatively for many years gets discovered after years of abuse and when it is discovered, the entity is put out of business in a short time causing chaos for the patients, providers, health plans, medical groups, and the regulators. A better system is needed.

### BRIEF DESCRIPTION OF THE DRAWINGS

The accompanying drawings are included to provide further understanding of the invention and constitute part of the specification. The drawings listed below illustrate embodiments of the invention and, together with the description, serve to explain the principles of the invention as disclosed by the claims and their equivalents.

FIG. 1 depicts a block diagram of a general view of the entities involved in the Healthcare Blockchain System, according to some embodiments.

FIG. 2 depicts a block diagram of Healthcare Blockchain System communicatively connected to an Analytics Engine, a deep learning and decision logic engine/Brain, an Application Programming Interface, and a plurality of existing healthcare systems, according to some embodiments.

FIG. 3 depicts a block diagram of a centralized deep learning and decision logic engine/Brain connected to a plurality of entities involved in the Healthcare of a patient and a blockchain network, according to some embodiments.

FIG. 4 depicts a block diagram of a system for capturing, storing, and reporting data using blockchain technology according to one of the embodiments of the present invention.

FIG. 5 is a flowchart of a Specialist Approval process, according to some embodiments.

FIG. 6 is a flowchart depicting member verification and appointment scheduling, according to some embodiments.

FIG. 7 is a flowchart depicting once cycle of operation of a patient service, according to some embodiments.

FIG. 8 is a block diagram for ensuring timely payments and verification of payment delivery, according to the embodiments of the present invention.

FIG. 9 is a flowchart of one exemplary process for ensuring timely payments and verification of payment delivery, according to the embodiments of the present invention.

FIG. 10 is a block diagram representing one embodiment of an automated dispute resolution process, according to according to the embodiments of the present invention.

FIG. 11 is a block diagram for performing error checking and predictive analysis claims, according to the embodiments of the present invention.

FIG. 13 is a flowchart of one exemplary interaction between the care provider and the payer system for processing a payment, according to the embodiments of the present invention.

FIG. 13 is a flowchart of one exemplary process used by the payer system to validate a claim and provide claim adjudication, according to the embodiments of the present invention.

FIG. 14 is a block diagram of an automated audit process, according to the embodiments of the present invention.

FIG. 15 is a block diagram of a computer system that can is used in operation of the centrally storing healthcare transactions, providing automated dispute resolution, performing predictive analysis on claims, performing an audit, automatically verifying care providers and their updated fees, posting healthcare transactions to blockchain and other features disclosed in the present invention, according to some embodiments.

While the embodiments of the application are susceptible to various modifications and alternative forms, specific embodiments are provided as examples in the drawings and detailed description. It should be understood that the drawings and detailed description are not intended to limit the embodiments to the particular form disclosed. Instead, the intention is to cover all modifications, equivalents and alternatives falling within the spirit and scope of the invention as defined by the appended claims.

### DETAILED DESCRIPTION

Systems and methods are disclosed for monitoring, recording, verifying, auditing, making suggestions by applying deep learning and artificial intelligence technology, and executing healthcare communications, decisions, and transactions using centralized storage and blockchain technology. The system and method use a plurality of devices, such as client computers, servers, mobile devices, computing devices, storage systems, and other handheld electronic devices for accomplishing the functions performed.

The system allows bi-directional communications and transactions between a Patient, their Healthcare Provider, the Healthcare plan provider, a Payer/Payer System, and a private, regulatory, or other government entity (hereinafter referred to as “Parties”) that process and reimburse healthcare providers, insurance providers, and healthcare facilities.

A Care Provider can be a primary physician, a doctor, a specialist, nurse, physician's assistant, clinic, laboratory, a medical facility, urgent care, hospice care, hospital, nursing home, rehabilitation center, a medical or laboratory technician or institution, pharmacy, home health care and related professionals, dentists, and other individuals or entities in the business and profession of providing medical care (collectively hereinafter referred to as “Care Provider” or “Healthcare Provider”).

These communications and transactions are both centrally stored as well as posted to a blockchain. The transactions relate to medical care provided by a Healthcare Provider for a patient having a health care plan. In addition, the transactions also relate to payment processing, audit, dispute resolution, booking appointments, determining health plan coverage, authorizing coverage and reimbursements, and credentialing Healthcare Providers.

The unique method of centrally storing as well as posting to a blockchain provides transparency to the parties involved in the process. Another benefit of this type of storing and posting is that it provides a tamper proof record of all events such that any dispute, error, of claim can be resolved by retrieving the stored and posted data. Further, the system engages the parties through an Application Interface (API) such that they can be part of the process and not a bystander.

The centralized control manager is connected to a central database and it utilizes the central database for storing healthcare data. The centralized control manager is operated through a processor. The processor executes instructions stored in the database and causes the centralized control manager to perform its functions.

One aspect of the invention is to allow communications between the parties, such as between patient, doctor, primary physician, specialist, a payer system, a health plan provider, and a government entity such as Medicare. As mentioned above, the communications are recorded centrally and stored in the blockchain allowing future retrieval. A centralized control manager is electronically and communicatively connected to the parties and allows bi-directionally communicate with the centralized control manager. Parties also communications directly with each other using the centralized and blockchain system.

The system also includes a deep learning and prediction engine that continuously enhances the overall system, provides suggestions, and predicts the outcome of future transactions. The system applies deep learning, artificial intelligence to a variety of date, such as historical data, crowd sourced data, data from regulatory agencies, and industry data for best practices, to improve system efficiency, accuracy, and minimize errors. Examples can be that the system can learn and prevent a transaction where a patient lives in and saw doctor in Georgia but is receiving a $30,000 wheelchair in Florida.

The system is also capable of performing automated dispute resolution. Since the data for all transactions are stored both centrally and in the blockchain, there is concrete evidence of all the communications and interactions between the parties. The system uses this data as evidence in analyzing and automatically resolving disputes between parties. For example, a dispute between a Payer and a Healthcare Provider on whether appropriate payment was made on a claim is resolved by retrieving data pertaining to the specific claim in dispute, obtaining all communications relating to the specific claim between the Payer and the Healthcare Provider, either obtaining authorized access to bank account for the Healthcare Provider or having them upload bank statements, and reviewing all the data in light of the dispute to then apply decision logic and resolve the dispute. In addition, the system also provides steps to perform the steps after resolution, such as transfer the payment based on the resolution to the Payer or engage the mailing processor to automatically send out mailings and checks.

Systems and methods for performing an audit are also described. The system uses unique approaches to retrieve claims from the central database. A sample size from the retrieved set of claims is then screened and selected to be audited based on a plurality of factors. A pass/fail result is then produced. The system then produces an audit note and summary of the audit. The system applies deep learning and artificial intelligence concepts to provide suggestions for enhancing audit efficiency and accuracy and minimizing future audit fails. When a party fails an audit, the system is capable of placing them on a corrective action plan. The audit is automated and the steps as well as the results are stored in the central database as well as posted to the blockchain.

Additional features of the present invention include allowing legacy systems to use the centralized storage and blockchain concepts by providing a downloadable plugin, applying unique methods of deep learning and artificial intelligence to healthcare transactions, managing conversations between parties, verifying members, verifying Healthcare Provider credentials, automating mailings to parties, providing bank verifications and payment verifications, using crowdsourcing methods to gather data for use in deep learning, booking appointments based on calendar mapping, and credentialing Healthcare Providers and updating their fees in real-time. The methods, systems, and functionality and features mentioned above as well as additional functionality and features of the present invention will be detailed and described below.

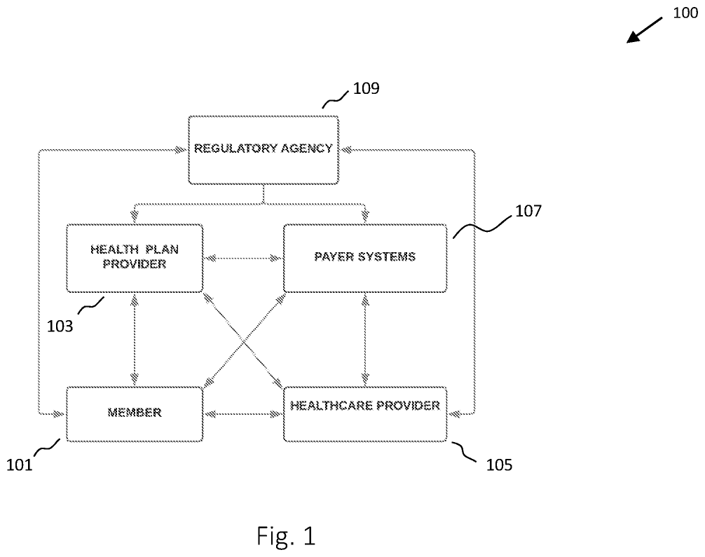

FIG. 1 is a block diagram of a general view of the entities involved in the Healthcare Blockchain System 100, according to some embodiments. The system includes a Member 101, a Health Plan Provider 103, a Healthcare Provider 105, Payer System 107, and a Regulatory Agency 109 (collectively referred to as “parties”). The member is a patient or an individual that received medical care. The member may be single individual or a primary or secondary member family or group.

The member of the system has a health care plan that provides for their medical care. The member is communicatively connected to one or more parties through an Application Programming Interface (API) that can be downloaded on their laptop, desktop, or mobile device.

The health plan provider 103 may be an institution such as Blue Cross, Blue Shield, Aetna, Kaiser, Cardinal Health, or United Healthcare. The health plan provider 103 typically offers a plurality of health care plans and the member selects a healthcare plan that suits their financial and medical needs. The risk and tolerance of each healthcare plan differ and the member selects the plan based on the estimation of the amount of medical care needed for the year and pays the monthly dues or a deductible towards the health plan in order to get medical coverage.

In some instances, the plans offered by the healthcare plan provider may be passed to them or prepared or regulated by a regulatory or government agency. A member needs to qualify for Medicare or other healthcare plans offered by a state or federal agency. For example, turning 65 years old is one way you become eligible for Medicare and another factor for eligibility is if you receiving Social Security Disability Insurance, State sponsored medical plan also determine the level of income, poverty, and need, before adding an individual to a government sponsored plan.

The function of a Healthcare Provider 105 is to provide care for the member's medical needs. These include patient diagnosis, health screening, laboratory work as well as Xrays or MRIs, specialist care, hospitalization, both short term and long-term care, surgical procedures, and any other care needed to address the patient's medical needs. The Healthcare Provider 105 and Member 101 communicate and interact not only for providing care but also for various procedural and administrative tasks that are needed to move the patient care forward. For example, one of these interactions could be to refer the member to a specialist and obtain approval from the Health Care Plan Provider 103 prior to referring to the specialist. The healthcare provider can also bi-directionally communicate with the regulatory agency.

A Payer System 107 is also part of the Healthcare Blockchain System 100. It is an entity other than the member that finances or reimburses the cost of health services. These entities include insurance carriers, third-party payers, health plan sponsors, employers, or Unions, and in some cases government entities. For example, some of the Payer Systems widely known are Hill Physicians Group, Blue Cross, Blue Shield, and Health net.

The Payer System 107 interacts with Member 101, Health Plan Provider 103, Healthcare Provider 105, and Regulatory Agency 109. The interactions between them include billing, accounting, payments, portioning of a bill to split the payment, reimbursement from the Regulatory Agency, and distribution of payment in accordance with contracts to Member 101, Health Plan Provider 103, and Healthcare Provider 105.

Payer system also obtain data from Member 101, Health Plan Provider 103, and Healthcare Provider 105 and perform the tasks of ensuring that the data is accurate, complete, and fair. Payer systems map the diagnosis of the patient with the medical codes provided to them by the Health Plan Provider 103 or the Healthcare Provider 105 to ensure that proper codes are entered. Payer system may also be compatible with various Electronic health records software and be able to obtain data from them that is authorized by the Healthcare Providers.

A Regulatory Agency 109 may also be involved in the Healthcare Blockchain System 100.

These Regulatory Agencies may include Center for Medicare & Medicaid Services (CMS), Department of Managed Health Care (DMHC), Department of Health Care Services (DHCS), Department of Health and Human Services (HHS), Department of Health and Human Services Office for Civil Rights (OCR), and any other healthcare related Regulatory Agency that comes under the State or Federal Government.

The role of these Regulatory Agencies 109 is to set health care regulations and standards that are needed for legal compliance, safety, and proper running of healthcare system in the United States. As such, they monitor Healthcare Providers, medical facilities, hospitals, and all other entities involved in the healthcare chain that provide patient care or are associated with billing, procedures, or management of healthcare services. They establish rules and regulations for the health care industry and provide oversight to ensure that health care organizations promote and provide quality and equal care without any fraud or discrimination. They also set the standards and pricing for payments for types of medical treatments and procedures such that patients are not overcharged.

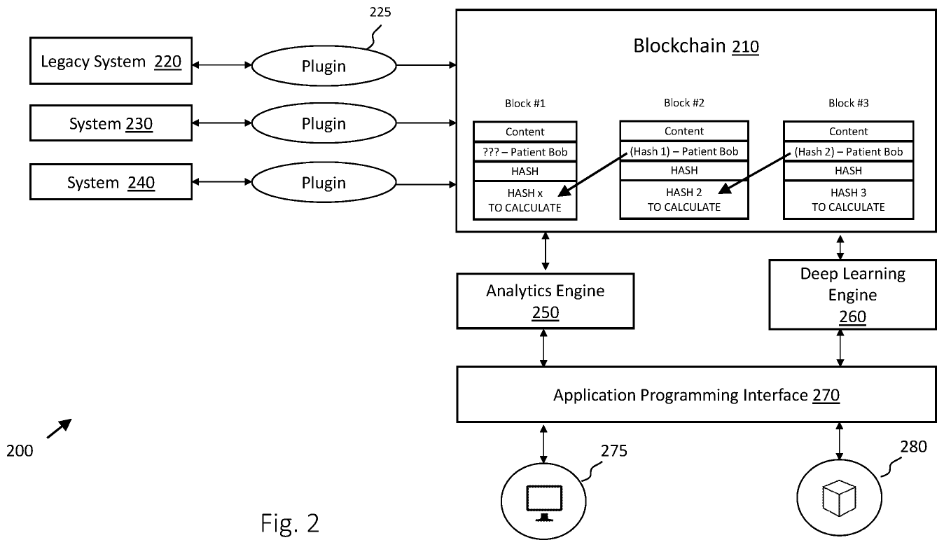

FIG. 2 depicts a block diagram of Healthcare Blockchain System communicatively connected to an Analytics Engine, a deep learning and decision logic engine/Brain, an Application Programming Interface, and a plurality of existing healthcare systems, according to some embodiments.

System 200 connects older and existing systems to a blockchain system 210 through plugins 225. Existing or older systems, such as Legacy Systems 220, Systems used by a Healthcare Provider (Provider Systems 230), and systems used by a Payer (Payer Systems 240).

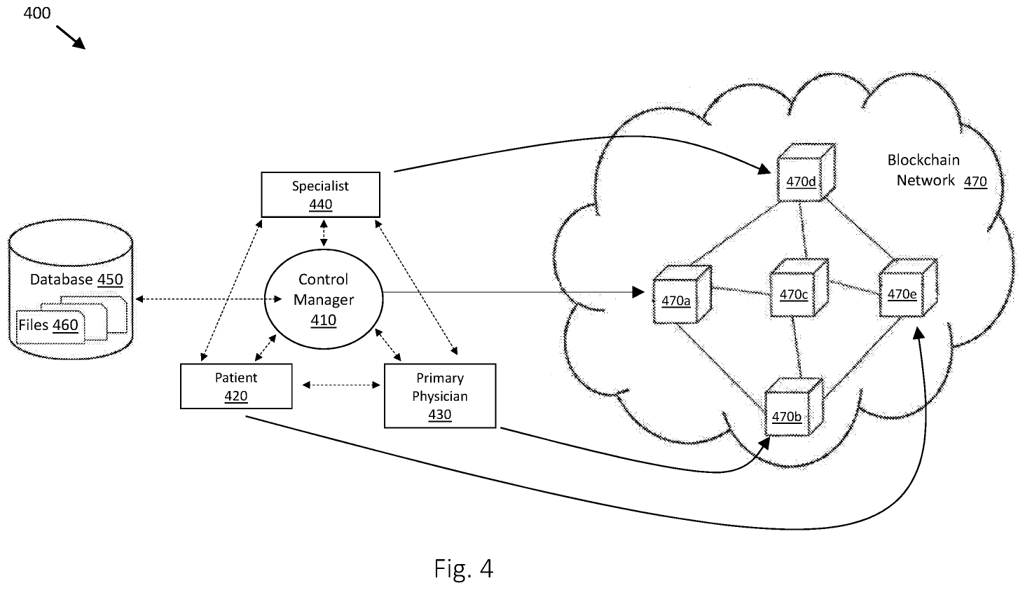

The blockchain system 210 includes a plurality of blocks. Each block includes content, header, hash, and data of the previous hash. The hash function basically takes input data of any length and creates a defined output of a fixed length. More specifically, it takes a string of random letters, also known as the digital fingerprint and produces a new hash. The hash of the new block, such as block 2, stores the hash of the previous block 1. These hashes generate cryptographic signatures that determine if the transactions are valid. Additional details of the blockchain system are described in FIG. 4.

The plugins 225 are downloadable files that can be downloaded on the legacy, payer, or Healthcare Provider system. Once downloaded, the plugins capture all the transactions that are performed by the systems and record the transactions into the blockchain 210. The plugins request and obtain data of each transaction, including the date/time of the transaction and the content of the transaction to record in the blockchain.

The plugins are designed such that they do not change the existing code of the legacy, provider, or payer systems. They get plugged internally and have two internal components, one which is specifically crafted to handle the data structures of existing system and the other component is responsible to authenticate and push the data into the cloud based blockchain.

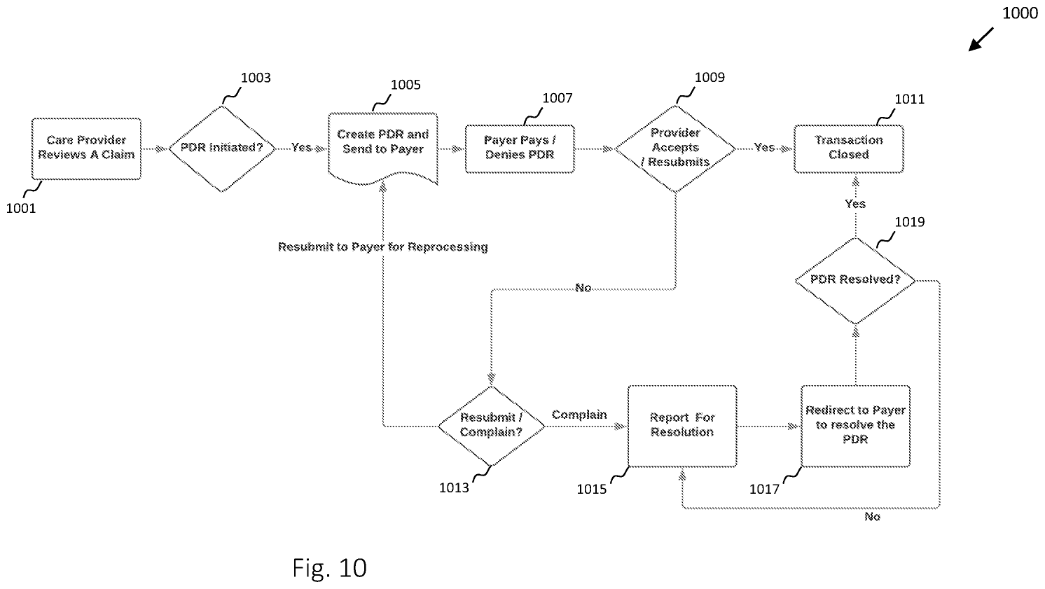

The system also includes an Analytics Engine 250 and a Brain 260 connected to the Blockchain system 210. The analytics engine 250 obtains the data recorded into the blockchain and analyses the data and parses the data to enhance the overall system 200 for future processing. The data is then used by machine learning modules (not shown in FIG. 2 and described further in FIG. 10) to predict claims, reduce errors, and suggest changes to data based on historical data stored in a database as well as other inputs from parties that are within and outside of the network. The suggestions made by machine learning platform are used in conjunction with the blockchain transaction data to annotate the data and advance to the next level of process with more accuracy and certainty, thus reducing the chance for human error.

The analytics engine 250 operates by obtaining real-time data thrown from the events and transactions in the blockchain. The analytics engine 250 routinely or on a set time interval collects data and metrics from the blockchain, then analyses the data to provide deep insights on a very large set of data. The analytics engine 250 also gets input from the Brain 260. The analytics engine visualizes these insights and represent them on a dashboard.

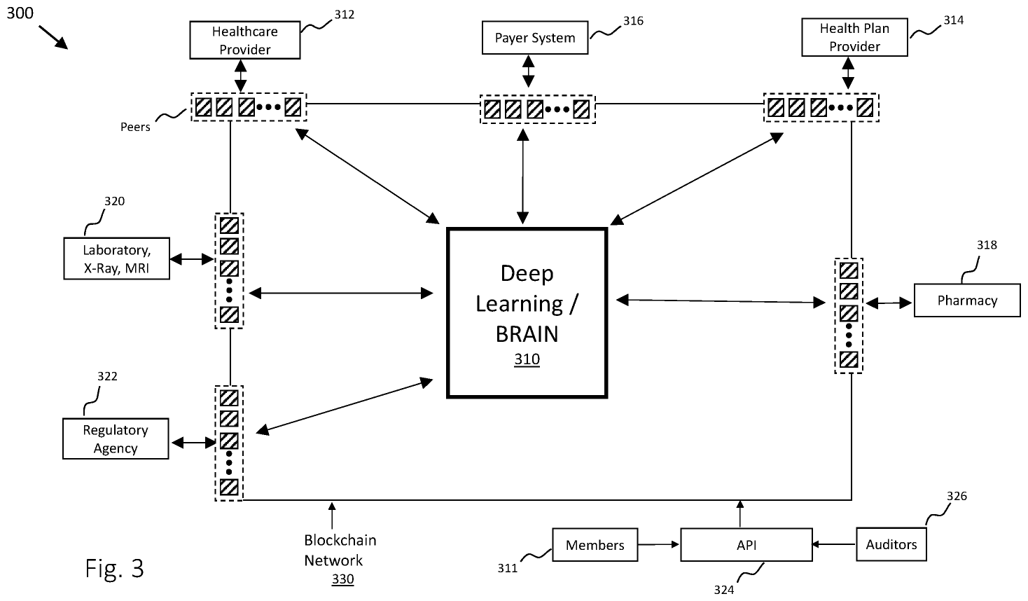

The deep learning engine 260, also referred to as the Brain, acts as the main logic, decision making engine for system 200. It stores data captured in a central database such that the data can be accessed by everyone connected to the network or everyone who is authorized to receive the data. Additional functions of the Brain are described in FIG. 3. The brain also learns from the data flowing through the system and constantly improves overtime as more sets of data are passed through. The brain helps in detecting the anomalies and potential discrepancies in the medical data and suggests the corrective measures for the same. It also helps in claim scrubbing, auto adjudication of claims and claim authorizations.

System 200 also includes an Application Programming Interface (API) 270. The API 270 communicates with a plurality of electronic devices, computers, servers, and databases to allow access to the data stored centrally as well as in the blockchain. For example, a member of the system, such as a Patient, can download an Application on their mobile phone and connect to the system through the API thereby allowing the Patient access to their records, communications between Healthcare Provider and other parties relating to their claims and medical treatment etc. The API also allows connections from a website 275 or a mobile application.

FIG. 3 depicts a block diagram of a centralized decision logic engine/Brain connected to a plurality of entities involved in the Healthcare of a patient and a blockchain network, according to some embodiments. The core part of the system is its centralized decision logic engine, also referred to as the Brain 310.

The system includes a plurality of parties. These parties include a member or members 311 (Patient), a Healthcare Provider 312, a Health Plan Provider 314, and a Payer or a Payer System 316. The system may also include additional parties such as a Pharmacy 318, a Laboratory or test taking facility 320, and a regulatory agency 322. As shown in the Figure, each party is connected to a plurality of peers. The blockchain network used by the system is comprised primarily of a set of peer nodes (or, simply, peers). Peers are a fundamental element of the network and they host ledgers and smart contracts, for example, each peer can hold copies of ledgers, copies of smart contracts, instances of the ledger, and instances of chain code. The peer nodes also provide a deliberate redundancy in a Blockchain Fabric network and avoid a single point of failure.

The regulatory agency or authorized regulatory agency can be Medicare & Medicaid Services (CMS), Department of Managed Health Care (DMHC), Department of Health Care Services (DHCS), Department of Health and Human Services (HHS), and Department of Health and Human Services Office for Civil Rights (OCR) or other authorized regulatory agencies.

Further, the system may also allow communication with Auditors 326 through an Application Programming Interface (API) 324 and be communicatively connected to a blockchain 330. Using the API, auditors can provide or access data, provide comments on a specific transaction, or post suggestions or industry standards that should be followed.

In one embodiment, the system functions to allow central storage of data that relates to healthcare of a member. The healthcare data includes patient's basic information and background, their medical history, medical diagnosis and claims related to the member, and processing of medical claims, payments, reimbursements, and interactions between the parties relating to all claim and auditable items concerning the member.

In one embodiment, a medical claim for payment is processed by the system 300. In this embodiment, several parties such as member, Healthcare Provider, payer, and health plan provider may take part in processing of the claim. The member receives services for which a Healthcare Provider needs to get paid. The payment may be either from the health care plan provider, member, or a mix of both. In some instances, the health claim provider may be a government entity, such as Medicare of a State sponsored health program.

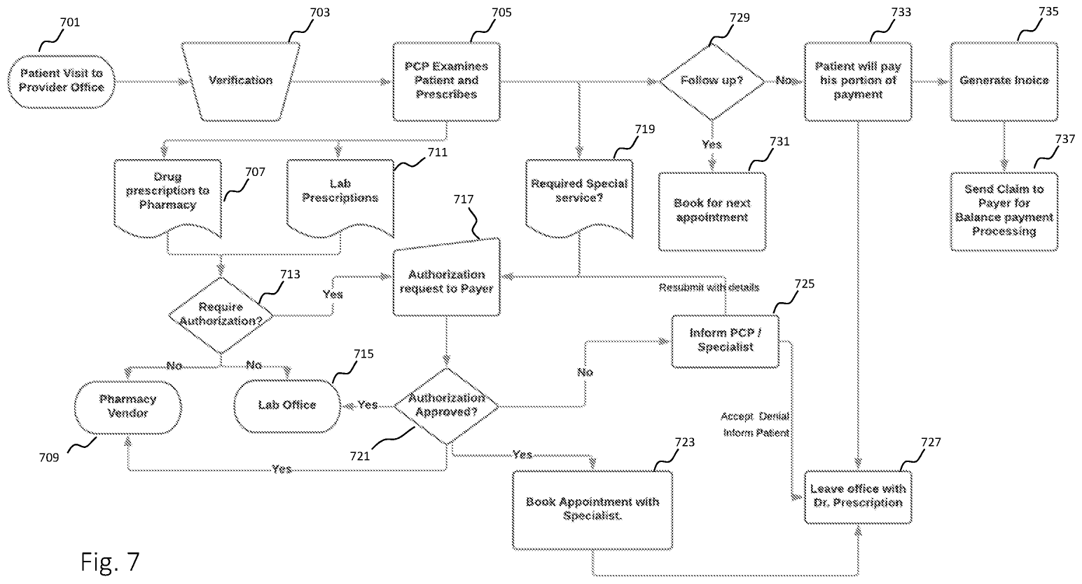

In this embodiment, once the member received medical care, the Healthcare Provider 312 submits a claim for payment to the Payer 316. The Payer 316 then evaluates the claim, matches the care received with the health plan coverage and pays the Healthcare Provider 312, some or all of the fees. In some instances, the Payer 316 pays a portion of the medical claim and requires the member to pay the remainder of the claim as negotiated by the contract formed when the member signed up for the health plan. Several alternative interactions and exchanges may occur between the parties before a payment is made (see further detail in FIGS. 7-12). The system 300, through its Brain 310, functions to record every interaction and transaction between the parties, including, dates and times of the transactions, details of the transactions, information exchanged through the transaction, and the ultimate outcome, e.g. payment, non-payment, portion of payment etc., time of payment, amount paid, parties paid etc.

The centralized approach allows all parties in the system 300 to view transaction and their details. In an alternative approach, parties relevant to the transaction or parties that have authorized access to the transaction may be the only entities that can view the transaction and the details. Each party relevant to the transaction may also raise a dispute, log a complaint, or simply have a need to audit the details of a transaction for payment or some other resolution. Since all the transactions, as that occur, are centrally logged by the Brain into a database or a third-party server, the party can simply retrieve the evidence and the log of the transaction and all related events and be also to expeditiously audit and resolve any issues. For example, if a claim was not authorized or not paid in time, the Healthcare Provider 312 may request from the Brain 310, or directly access, all the relevant events that led to the delay of payment such that they can query the payer and resolve or point out the delay. Likewise, a Payer 316 may be able to provide proof of a check made to the Healthcare Provider 312 if such a check went missing thereby using evidentiary proof that a check was in fact mailed and it was not a delay on their part. Whatever the case may be, whether it's a delay, improper payment, or other issue, the centralized process allows transparency such that each party can review the transaction and all related events and be able to audit to retrieve the required details. Further, when a party raises a dispute or a complaint, all the involved parties to that transaction may be informed and be able to review the transaction details such that they may either comment or simply monitor the audit.

As mentioned above, the system 300 is also connected to a blockchain network. In this embodiment, in addition to a centralized operation and storing process, the system also utilizes a blockchain network and records each transaction into a block of the blockchain network. The system is able to match all the entries into various blocks of the blockchain and flag anytime a block does not match another block in the chain signifying that various parties have a different recollection of the transaction and there may be an error associated with the data.

In one embodiment, the system 300 uses a certain type of blockchain framework that allows communication and interaction between multiple agents. This approach allows multiple agents to work on a solution that may be difficult for a single agent to solve. The approach results in a better-informed solution where every agent gets an opportunity to provide relevant input on a transaction. Agents can also develop their own applications and services and integrate them into the system thereby allowing other agents access to their applications and services.

One exemplary use of the system 300 connected to the blockchain network is for credentialing physicians, specialists, and other medical staff on a regular and real-time basis. A medical practice, facility, clinic, hospital, nursing home, or urgent care often undergo change of staff, change of specialties, and change in services offered. Many medical program, such as Medicare and others, require a physician to be credentialed and at optionally their profile listed on the webpage of the medical facility. Credentialing is a process where a Healthcare Provider, such as a physician or specialist undergoes an evaluation that reviews their education, training, residency and licenses and determines if they are in good standing to practice medicine. The process evaluates their qualifications and practice history. It also evaluates their certifications issued by a board in the doctor's area of specialty and any educational or practice updates.

The present invention allows a medical professional to update their education, training, residency, licenses, and certifications, and any other update relevant to their practice and records the update or chain in the blockchain. Other authorized entities, such as a medical board, peer reviewers can also add to the credentialing and all the data is recorded to the blockchain thereby allowing all entities connected to the blockchain to be able to see the update, change, or additions by another entity often in real time. This alleviates duplicate efforts of each entity that requires the credentialing to obtain such data from the doctor or the medical facility to update their own records. For example, an insurance carrier needing to verify credentialing of a doctor will have access and most up to date record through the present invention's use of blockchain and be able to verify the credentials.

FIG. 4 depicts a block diagram of a system for capturing, storing, and reporting data using blockchain technology according to one of the embodiments of the present invention. System 400 generally includes a control manager 110 (referred to herein as “node 410” or “control node 410”), which can be considered a computer node. Although only a single node 410 is shown, a plurality of such nodes that form the network are also contemplated by the present invention. As further shown, the system 400 includes a Patient 420, a Primary Physician 430, and a Specialist 440 (which are also considered as nodes of system 400). These nodes are configured to issue transactions or events to control node 410 as described in detail below. Each of the nodes of system 400 form part of a distributed storage system for one aspect of a healthcare blockchain system.

Although a Patient 420, a Primary Physician 430, and a Specialist 440 are shown, the present invention contemplates the same methodology for transactions between other entities of the present invention as shown in FIGS. 1-3 in this application, e.g., Healthcare Provider, Payer, Payer System, Health Plan Provider, Pharmacy, Laboratory, Member, Clinics, Hospitals, Auditors, Regulatory Agencies, and any other authorized entities that take part in the overall healthcare process for delivering, managing, administering, or authorizing healthcare for a patient. However, for exemplary purposes, FIG. 4 focuses on the interactions, transactions, and events between a Patient 420, a Primary Physician 430, and a Specialist 440.

According to one embodiment, the Patient 420 is digitally recognized in the system 400 through their digital profile that may be stored on an electronic device or the database 450. The Patient 420 can be communicatively coupled to one or a plurality of Control Manager nodes 410 that are operators for providing services, recording events and transactions between connected parties (e.g. Patient, Primary Physician, Specialist, Database, and Blockchain Network). Each of the connected nodes can be any type of conventional electronic or computing device having a processor, such as a personal computer, server, laptop, smartphone, tablet, smart watch etc.

In this embodiment, the Patient 420 is configured to access one or more nodes in the blockchain network 470 in order to validate the service obtained from control node 410. For example, the Patient or Patient Node 420 is communicatively coupled to Control Manager 420, Primary Physician 430, Specialist 440, and Node 470a.

A Primary Physician 430 can be communicatively coupled to one or a plurality of Control Manager nodes 410 that are operators for providing services, recording events and transactions between connected parties (e.g. Patient, Primary Physician, Specialist, Database, and Blockchain Network). In this embodiment, the Primary Physician 430 is configured to access one or more nodes in the blockchain network 470 in order to validate the service obtained from control node 410. For example, the Primary Physician or Primary Physician Node 430 is communicatively coupled to Control Manager 410, Patient 420, Specialist 440, and Node 470b. Although a Primary Physician 430 node is depicted as an example, the node can be any Healthcare Provider.

A Specialist 440 can be communicatively coupled to one or a plurality of Control Manager nodes 410 that are operators for providing services, recording events and transactions between connected parties (e.g. Patient, Primary Physician, Specialist, Database, and Blockchain Network). In this embodiment, the Specialist 440 is configured to access one or more nodes in the blockchain network 470 in order to validate the service obtained from control node 410. For example, the Specialist or Specialist Node 440 is communicatively coupled to Control Manager 410, Patient 420, Primary Physician 430, and Node 470d. The Specialist can be a medical professional or another medical individual, entity, laboratory, or hospital that typically requires a referral from the Primary Physician or Caregiver in order to be able to allowed to see the Patient and be covered by their medical plan. Although not shown in this Figure, a Payer may also be involved in a transaction between Patient 420, Primary Physician 430, and Specialist 440 in cases where a coverage or a payment related authorization is required prior to providing the referral or in assessing whether the referral would be covered by the health plan.

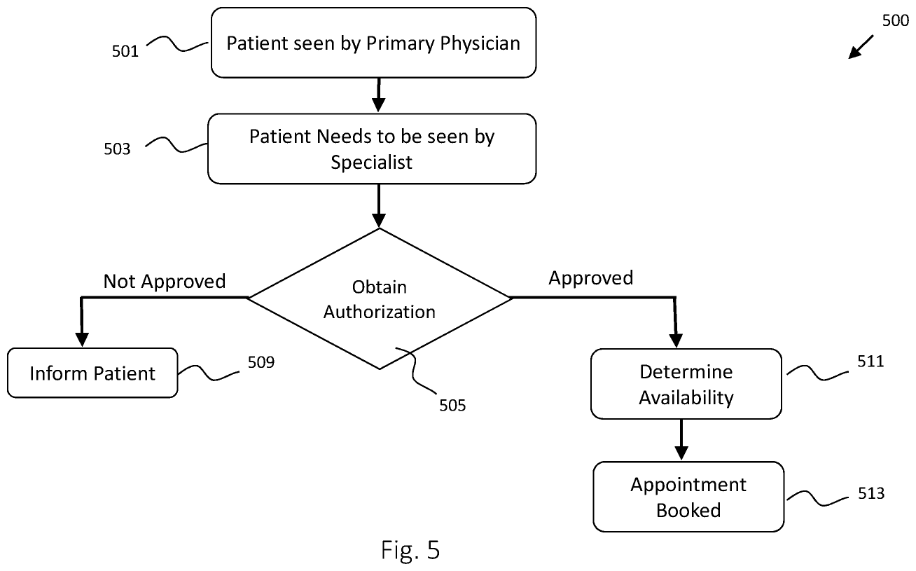

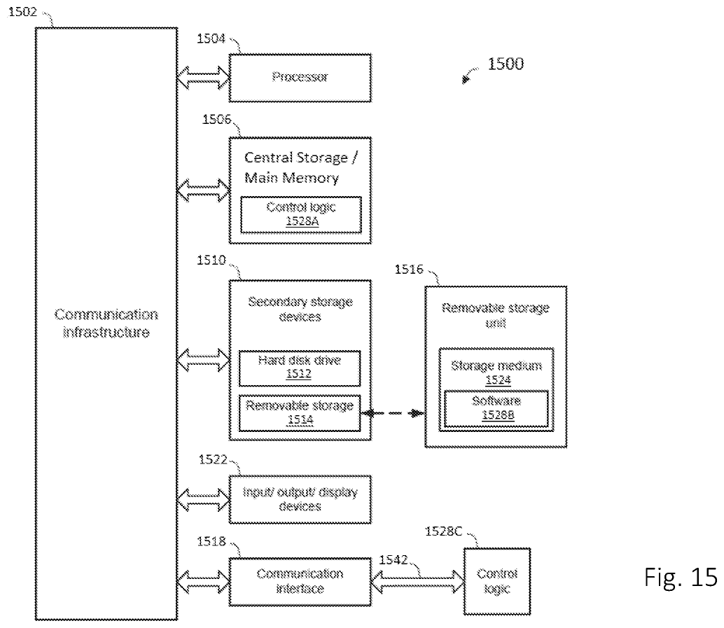

Additional details and applications of the control node 410 will be discussed below in FIGS. 5, 8, and 10, but generally the node 410 is configured to record events and transactions relating to the medical services and medical care provided to the Patient through their Primary Physician or Specialist. The node 410 also manages discussions, digital traffic, approvals, referrals, and other interactions between the physician and specialist, including discussions of reimbursements, patient billing, patient coding, scheduling, visitation, diagnosis etc. Node 410 obtains all such data and reports it to a node of blockchain network 470. Similar to other nodes, control node 410 may in general be any type of computing device, such as a laptop, server, a desktop, a tablet, or a mobile phone. Exemplary hardware details of the control node 410 will be described below in FIG. 15.

In addition, system 400 further includes database 450 and blockchain network 470. According to the exemplary aspect, the database 450 can generally include hardware and software components configured to manage various storage resources within the computing environment. For example, the database 450 can include one or more data storage devices (e.g., hard disk drives, optical drives, magnetic tape drives, Solid State Drives, NAND Flash Storage Drives, 3D XPoint Drives, or Microwave or Heat Assisted Magnetic Recording Drives and/or the like) and storage management software that provides an interface to the one or more data storage devices.

The database 450, which can be a data cloud storage service according to one aspect, facilitates temporary and/or permanent storage of computer data, including data files 460 of node 410. According to the exemplary aspect, the computer data of data files 460 may be any type of electronic, digital data generated and/or stored by a computer. For example, the computer data can represent text data, executable program code, audio, video or image data, or any other type of digital data. In the exemplary aspect, these data files 460 correspond to events and transactions relating to healthcare of the Patient 420 that are provided either by the Primary Physician 430 or the Specialist 440 or any interaction, event, or transaction relating to the specific Patient 420. Moreover, as will be discussed in detail below, the node 410 is configured to manage, review, record, and obtain data relating to healthcare of the Patient and store it in database 450 and transmit updates (i.e., transactions or changes) to this information to blockchain network 470. Additional information can be stored include the file name, size in bytes and storage transaction number of the file.

According to an exemplary aspect, the blockchain network 470 can be an existing (public or private) distributed network formed from a plurality of nodes or computers 470a-470 e. In one embodiment, the blockchain network includes parties (or nodes or computers) that are authorized to receive, review, or hold confidential healthcare information specific to the Patient 420. For example, in one embodiment, the parties (or nodes or computers), are those that are allowed under HIPAA regulation to receive patient confidential information.

The blockchain creates a history of data deposits, messages, or transactions in a series of blocks where each block contains a mathematical summary, called a hash, of the previous block. In turn, this process creates a chain where any changes made to a block will change that block's hash, which must be recomputed and stored in the next block. This changes the hash of the next block, which must also be recomputed and so on until the end of the chain. One aspect of this Hash function is shown in FIG. 2 where the next block stores data from the previous hash and so on.

In the exemplary aspect, blockchain network 470 maintains a continuously-growing list of data records hardened against tampering and revision and is composed of data structure blocks that exclusively hold the data received from the node 410 (and other nodes in the system). In particular, after any changes are executed by operator of node 410 to any of files 460, the node transmits such data (and related data or metadata) to the blockchain network 470. The blockchain network 470 records this transaction in a block and confirms when and in what sequence the data transactions enter and are logged in the existing blockchain. Preferably, every node (e.g., computers 470a-470e) in the decentralized system has a copy of the growing blockchain.

This design avoids the need to have a centralized database managed by a trusted third party. Moreover, each of the nodes 470 a-470 e can validate the data, add hash values to their copy of the blockchain and then broadcast these additions to other nodes in accordance with existing blockchain methodologies. In general, different blockchain networks have different formats for descriptions of transactions. Thus, while all blockchain networks are generally configured to include hash values of the file, other fields can vary from network to network. In one aspect, node 410 itself forms a node within blockchain network 470 (e.g., computing device 470 a and node 410 are the same computing device forming a node in blockchain network 470).

Similarly, Patient 420 forms another node within the blockchain network 470 (e.g., computing device 470e and Patient 420 are the same computing device forming a node in blockchain network 470). Primary Physician 430 forms another node within the blockchain network 470 (e.g., computing device 470b and Primary Physician 430 are the same computing device forming a node in blockchain network 470). Specialist 440 forms another node within the blockchain network 470 (e.g., computing device 470d and Specialist 440 are the same computing device forming a node in blockchain network 470). As such, each of the solid arrows indicate that the control node 410 and computing device 470 are the same node.

According to the exemplary aspect, each of the components of system 400 shown in FIG. 4 is configured to transmit data across a network. As shown, the exemplary communication is illustrated as two-way dashed arrows. The applicable network can be any network for communicating data and data operations and can include a communication system (not shown) that connects the various components of the system 400 by wire, cable, fiber optic, and/or wireless links facilitated by various types of well-known network elements, such as hubs, switches, routers, and the like. It should be appreciated that the network may employ various well-known protocols to communicate information amongst the network resources. In one aspect, the network can be part of the Internet or intranet using various communications infrastructure such as Ethernet, WiFi and the like.

System 400 is applied to events and transactions involving healthcare of a patient. Since numerous events and transactions occur in providing healthcare to a patient, the blockchain system 400 is used to review, monitor, and record in blockchain all the relevant events that validate, expedite, and allow auditory controls over healthcare processing. FIGS. 5-7, 9, 11, and 12 are some examples of the types of events and transactions that use FIG. 4's system.

FIG. 5 is a flowchart depicting a Specialist Approval process, according to some embodiments. The process uses the system shown in FIG. 4 as well as some or all of the additional elements shown in FIG. 1.

At Step 501, a Patient is seen by a Primary Care Physician. The event is recorded in blockchain with details of the visitation. The event may include dates, times, diagnosis, medical codes, and other relevant information about the patient's visit. The event is also communicated to Control Manager 410 for recording in block 470a. The Primary Care Physician also records the event in block 470b. The contents of blocks 470a and 470b match. The patient may also record the event in block 470e. If there is a discrepancy in the details of the event between blocks 470a, 470b, or 470e, then the blockchain is disrupted or an error message is sent through the system.

At Step 503, if the Patient needs to be seen by a Specialist, then the Primary Care Physician or their office sends a request to all requisite authorities to determine whether a specialist is covered by the Patient's health plan. These requisite authorities may be health plan provider, payment system, or regulatory agency. The sending of the request is logged in blockchain. The information logged includes dates, times, and details send by the Primary Care Physician to the requisite authorities in an attempt to obtain verification of eligibility or approval to see a specialist. In certain cases, the law, regulatory agency, of business practices require the sending of such request within a certain time period from the date of the patient visit to the primary care physician. The details and the timestamp of the request is both logged in the blockchain and sent to the Control Manager 410. The Control Manager records the details in files 460 and stores them in the Database 460. The Control Manager 410 also propagates the message to the nodes in the Blockchain Network 400.

The Control Manager 410 may be programmed with rules and conditions. For example, one of the condition may be that the request for verification for a patient to determine whether their health plan covers a referral to the Specialist needs to be sent within X days, e.g. 12 hours, 1 day, 3 days, or other time frames. The time window may be programmed into the system either by user preference or it may be regulated by laws and regulations governing healthcare processes. In this scenario, of the request is not sent within the allotted time specified in the rules/conditions in the Control Manager, then the Control Manager may have sent out an alert to one or more electronic devices alerting them of the delay. These electronic devices may inform the Patient, the Primary Care Physician or their management, Health Care Plan Provider, or Regulatory Agency. The system may be programmed to provide multiple alerts, some which may include internal deadline and some which may be of higher visibility for missing the deadline.

At Step 505, the system 500 responds with a decision, i.e., whether the patient is eligible to see a specialist. Eligibility basically means that the health plan used by the Patient will cover the costs for seeing the Specialist, otherwise, the patient would have to pay on their own out of pocket. The response also needs to be sent within a certain amount of time. As mentioned earlier, the time window may be programmed into the system either by user preference or it may be regulated by laws and regulations governing healthcare processes. When it relates to Medicare or other government sponsored medical program, the response time must be within a certain amount of time that is regulated by the government.

Once the determination response is received, it is recorded into the blockchain. In this scenario, the responding party, which may be the Health Care Plan Provider and/or the Payment System, would record it into blockchain that a response was sent. The receipt of the response by the Primary Physician would also be recorded into the blockchain. Further, the Control Manager will also record the receipt of the decision into the blockchain.

If the decision of eligibility is not received within the allotted time specified in the rules/conditions in the Control Manager, then the Control Manager can send an alert to one or more electronic devices alerting them of the delay. The system 400 promotes expeditious processing and acts to prevent delay or have parties that delay be accountable for missing the deadlines.

At Step 509, a decision that the Specialist is not covered is relayed to the Patient. Once again, the decision, the date/time it was relayed to the Patient, the date/time it was relayed to the Primary Healthcare Provider is logged into the blockchain. At this point, the Patient may either chose to pay for the specialist themselves or appeal the decision. The requisite record and data needed for Appeal would be readily available through the blockchain as well as Database 450 and files 460 that have recorded all the interactions and transactions between the parties.

At Step 511, a positive decision indicating that the Specialist is covered by the health plan is conveyed to the Patient through the API. Once again, the decision, the date/time it was conveyed to the Patient, the date/time it was conveyed to the Primary Healthcare Provider is posted to the blockchain. The data is also reported to the Control Manager who then records the data in the files of the central database.

It may also be the case that the Specialist is partially covered by the Health Care Plan. In such instances, the Payer Systems may also get involved and together with the decision, or in a separate communication, information on what portion is covered, what amount is paid by the Health care Plan Provider and what is the Patient responsibility would also be provided to the Patient. The details of the communication, the type of coverage, the decision itself, and the payment and billing details would also be logged into the blockchain by multiple parties (e.g., Health Care Plan Provider, Payer System, Specialist, and Control Manager). At this point, the Patient may either chose to partially pay for the specialist themselves or appeal the decision or check the payment split with the health care plan to determine if the plan is required to cover the partial amount. The requisite record and data needed for checking or appealing would be readily available through the blockchain as well as Database 450 and files 460 that have recorded all the interactions and transactions between the parties.

Once the positive decision has been made, i.e., a decision that the Patient is eligible to see the Specialist and the Health Care Plan either partially or fully covers the costs, then the system checks for availability of the Specialist. In some instances, a Healthcare Provider may have made a referral within their own network and it turns out that such a specialty is not available in their group. In such cases, the error is reported back to the referring Healthcare Provider to make another referral.

Unrelated to this application, in real life, there have been records of Patient Referrals and Billings when such a specialty is not available in a group and Patient being given the runaround or having to wait until a Specialist becomes available. The present invention acts as a deterrent to such run arounds or referrals within the same medical group when such specialty does not exist in the group to ensure the referral is proper. It does so by logging all details in a blockchain and cross-matching previously loaded Specialist profiles to determine whether such a specialty existed at the time of referral and further maps the availability of the Specialist to accommodate the Patient within the time frame the Patient needs to be seen by the Specialist. The blockchain data will reflect whether the availability as well as the specialty existed and whether referral was appropriate to refer within the own group. Since the data cannot be changed or manipulated in the blockchain without affecting the entire chain, of an improper referral has been made, or an improper bill or reimbursement request has been made, then the record acts as evidence of the wrong referral.

At Step S013, a match between the Specialist's availability and the Patient availability is made and an appointment with the Specialist is booked. The booking is recorded in the central database and posted to the blockchain.

All the events, transactions, communications, patient relevant data, process of requesting, verifying, responding, checking availability and booking is all logged or recorded into the blockchain by all the parties that are involved in this interaction. Further the Control Manager would also log the details. As such several blocks in the blockchain would have the same data. All the blocks would contain a hash, of the previous block, which also reports the same data, e.g., the type of eligibility decision, or the date the request was sent or received etc. The authenticity of the data is obtained through this process creating a chain of hashes where any changes made to a block will change that block's hashes well as the whole chain. As such the transactions provide transparency as well as accountability for all the parties involved in the process.

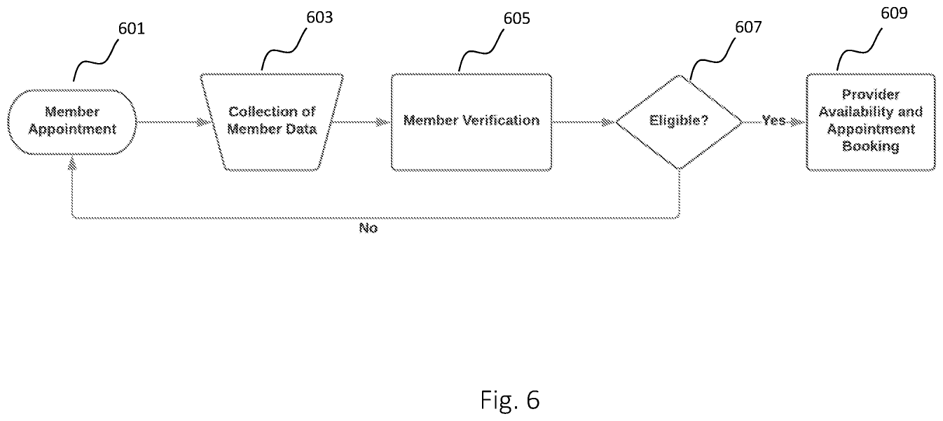

FIG. 6 is a flowchart depicting member verification and appointment scheduling, according to some embodiments. The process uses the system shown in FIG. 4 as well as some or all of the additional elements shown in FIG. 1. Additionally, process described in FIG. 6 is another example of the process described in FIG. 5. This exemplary process illustrates the member verification, data collection, eligibility and booking with a Primary Care Physician.

The member, who is a patient or an individual needing medical care, books an appointment with a Healthcare Provider at 601. The booking of the appointment is recorded in the blockchain.

At 603, the member data is collected by an individual or an automated system over the phone. The member data may also be imported from either a previous record or from another medical facility upon patient authorization. The member data may include some basic details that are general to the member as well as any specific medical health issues that they need to address in the current visit with their Healthcare Provider. The basic information may include date of birth, social security number, address, and emergency contact. Most extensive information may include past health record and medications, past diagnosis, allergies, labs or test results or other items specific in understanding the patient history. The current medical issues, such as the present medical condition, vitals etc. may also be obtained. All the obtained information will be recorded in the database and its files as shown in FIG. 4 as well as some or all of the data will be provided to the blockchain and accessible to other users of the blockchain either on a need to know basis or to everyone who is authorized to have confidential patient data. Various levels of security may also be created which unlock the various types of data obtained to parties in the blockchain. For example, certain data may be accessible only to the Healthcare Provider and not the health insurance, or certain data may be obtained by only the regulatory agencies.

At 605, the member is verified by checking the member eligibility with their respective health plans. The result of the eligibility will determination at 607 is recorded in the blockchain. A negative decision is reported to the member whereas a positive determination of eligibility results in step 609 where the provider availability is determined and an appointment is booked. All the transactions are recorded in the blockchain.

FIG. 7 is a flowchart depicting once cycle of operation of a patient service, according to some embodiments. The process uses the system shown in FIG. 4 as well as some or all of the additional elements shown in FIGS. 1 and 2.

At Step 701, a patient visits a Healthcare Provider's office or facility. The facility may be a clinic, hospital outpatient care, urgent care, or a nursing home. In some instances, the patient may be visiting a test facility to get an X-ray. MRI, or a blood test done.

At Step 703, the Patient is verified by the centralized blockchain system. The verification includes the Healthcare Provider sending an electronic query to the control manager 410. In response, the Control Manager 410 accesses a centralized database 450 to retrieve the patient's health plan information. The control manager 410 may also send a request to the health care plan provider to determine patient health plan verification. In either case, whether the data is retrieved from the central database or the health care plan provider, the data would verify whether the patient is currently enrolled and in good standing such that the health plan provides coverage to the patient for their medical care. Verification would also include details of any copay that the patient needs to pay to the Healthcare Provider, details of coverage, any payment sharing obligations for the patient, and deductible and out-of-pocket coverage as well as their current standing in meeting the deductible and out-of-pocket coverage.

The query, the response, and the authorization for verification are logged by the Control Manager 410 in the central database. Alternatively, the Healthcare Provider, Control Manager, and the Health Plan provider would record the query and the response, including the date and time of the query, the date and time of the response, the content of the response in a block of the blockchain network. In this scenario, each entity, i.e. the Healthcare Provider, Control Manager, and Health Plan Provider, would use separate blocks to record the data. The recording provides transparency and ensures that all the entities recorded the same data such that there are no discrepancies or misunderstanding in the verification or the plan coverage.

At Step 705, the Healthcare Provider examines the Patient. The Healthcare Provider may also prescribe medication, a test, or a follow up based on the patient's medical need. The Control Manager 410 records the examination date, time, and diagnosis details in the centralized database 450. Further, both the Healthcare Provider and the Control Manager 410 post the event details to the blockchain.

At Step 707, the Healthcare Provider prescribes a medication for the patient. The medication may be pills, liquid medication, or other medical remedy that is typically carried by a pharmacy. Once the medication prescription has been made by the Healthcare Provider, the prescription and the details of the prescription along with the details of the receiving party (which is the pharmacy) is recorded in centralized database 45 by the Control Manager 410. The Healthcare Provider, Control Manager 410, and the Pharmacy post the event details to the blockchain. The posting and recording have several benefits, among which are transparency as well as fraud protection preventing a patient to fulfill the same medication, more than once.

At Step 709, the Pharmacy vendor, such as Walgreens, CVS, Walmart or Rite-Aid, process the prescription and prepare the medications accordingly. All the drug prescriptions and their fulfillment are also centrally recorded as well as posted to the blockchain. Alternatively, in some cases where specialty drugs or medications are prescribed, the Pharmacy vendor may need prior authorization from the Payer for filling the medications. For such cases, a prior authorization is sought and medications are filled only upon approval.

At Step 711, the Healthcare Provider prescribes a lab test to be performed on the patient. The lab test may be a blood test, urine test, X-ray, or a variety of medical tests that are required to determine patient health. Once the laboratory prescription has been made by the Healthcare Provider, the prescription and the details of the prescription along with the details of the receiving party (which is the lab) is recorded in centralized database 450 by the Control Manager 410. The Healthcare Provider, Control Manager 410, and the Lab post the event details to the blockchain. As mentioned earlier, the recording and posting provide transparency and ensures that all the entities recorded the same data such that there are no discrepancies or misunderstanding and provides evidence that a lab test was ordered by the Healthcare Provider and also includes the details on the type and nature of the lab test.

At Step 713, the laboratory determines if an authorization for the test is required. If not required, then the laboratory performs the test at 715 and reports the results to centralized database 450 through the Control Manager 410 and also posts it to the blockchain.

However, if at 713, the laboratory determines that an authorization for the test is required, then at 717, an authorization request is sent to the Payer and once it is authorized at 721, the test is performed at 715. Alternatively, if the Payer does not approve the authorization request at 717 and 721, then the Healthcare Provider is informed at 725. At this point the Healthcare Provider is left with the option of either resubmitting the laboratory request for authorization or accepting the denial of authorization and providing the patient an alternative prescription for medication in lieu of the laboratory test. It is also contemplated that some authorization may require the patient to see a specialist and if so, the system books an appointment at 723 and the details are once recorded in the centralized database 450 and posted to the blockchain by the Control Manager 410.

Referring back to Step 705, there may be cases in which the Primary Care physician may refer the Patient to a specialist. In such cases, Steps 719, 717, and 721 may be followed to get authorization from the Payer for the Patient to be able to see the Specialist, the cost of which would be approved by the Payer. The Payer would essentially be following the rules and conditions in the Patient's health plan to determine if cost of such a Specialist or service is covered by the health plan. If approved and authorized, then the Patient may be booked for a Specialist appointment at 723, if not authorized then the Primary Care Physician would be notified at 725 at which point the Primary Healthcare Provider is left with the option of either re-submitting the request and explaining the need for the Patient to be seen by the Specialist or accept the denial of authorization and providing the patient alternative. The denial, the reasons for referral, the reasons for denial, along with explanations and other data communications between the Primary Healthcare Provider, Patient, Specialist, Payer, and Health Plan Provider while trying to obtain approval and authorization are once recorded in the centralized database 450 and posted to the blockchain by the Control Manager 410.

At Step 705, another alternative may be that the Patient needs to follow up with the Healthcare Provider at 729. In this case, the system would book an appointment with the Healthcare Provider at 731. In so doing, whether its booking at appointment at 731 or at 723, the system interacts with the Healthcare Provider and their information technology (IT) system to obtain the Healthcare Provider's availability and books the appointment for the Patient. Since the Patient is also an interactive member of the system, the Patient through an API, such as API 270 in FIG. 2 or API 324 in FIG. 3, can provide their own availability and preferred dates for the appointment. The system would then do an automated calendaring match and book an appointment that matches the Healthcare Provider's open schedule with the Patient's availability. The system may also operate in real-time and obtain real-time data for any cancellations and changes in the Healthcare Provider's schedule and in real-time book appointment based on matched schedule openings.

In some scenarios, such as at 733, the Patient will be required to pay their portion of the bill. This may be due to a deductible they need to cover for the health plan, a percentage that is Patient's liability based on the health plan selected or simply because the coverage is not approved by the health care plan. There may also be copay required.

At Step 735, an invoice, such as a Super Bill is generated. This bill is essentially an itemized form used by Healthcare Providers to detail out the medical services provided to the patient and is used by the Healthcare Providers for submission to health insurance and payers for reimbursements.

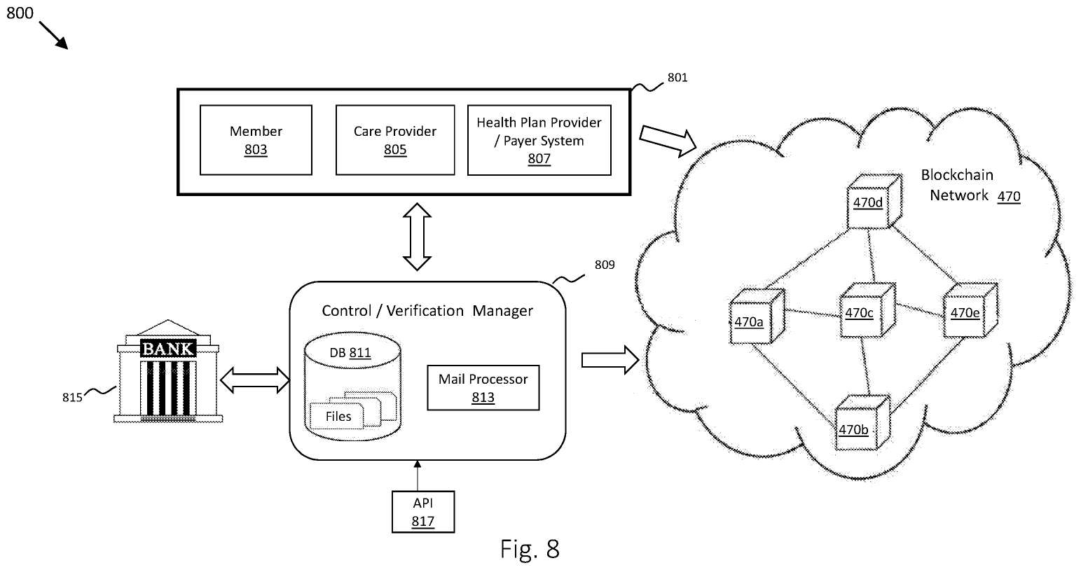

At Step 737, based on the health plan coverage, a portion of the bill will be sent to the Payer and the remaining portion, which is the Patient responsibility, would be mailed out to the Patient using a Mail Processor 813 (described later in FIG. 8).

FIG. 8 is a block diagram for ensuring timely payments and verification of payments delivered, according to the embodiments of the present invention. The system 800 includes parties 801 that include Member 803, Healthcare Provider 805, and Payer System or Health Plan Provider or both together 807. The system also includes a Control/Verification Manager 809. A database 811 and a Mail Processor 813 are part of the Control/Verification Manager 809. The system is communicatively connected to a Bank 815 or Financial Institution or a Clearing house. Further, the system is connected to a blockchain network 470 having a plurality of nodes 470a-e.

Parties 801 interact with each other and communicate to process a payment claim. For example, the member 803 receives medical care from the Healthcare Provider 805 and when the care is completed, the Healthcare Provider generates a medical code that is invoiced for payment. The payer system 807 then processes the payment in accordance with the health plan. All the transactions, communications, and events are recorded into the blockchain.

The Control/Verification Manager 809 acts as the middleman in processing all the claims and ensuring that payment is timely and made to the member in accordance with the health plan. In so doing, the Control/Verification Manager 809 writes all events to the blockchain 470 and also stores the events and relevant data in the files of the Database 811.

The Control/Verification Manager 809 also includes a Mail Processor. It functions to mail out notices and paychecks to the member. The mail processor is managed by the Control/Verification Manager 809 so that the payment amounts, the dates of payments, adherence and proper following of the payments in accordance with the health plans are verified and mailed out to the member. The Control/Verification Manager 809 also has the ability to verify transactions with the bank 815, These include, verification that a check has been requested, date it was sent, and electronic payment sent through the wire, verification of the check being cashed or not cashed, and all relevant financial date with respect to the check processing and cashing or receipt by the member.

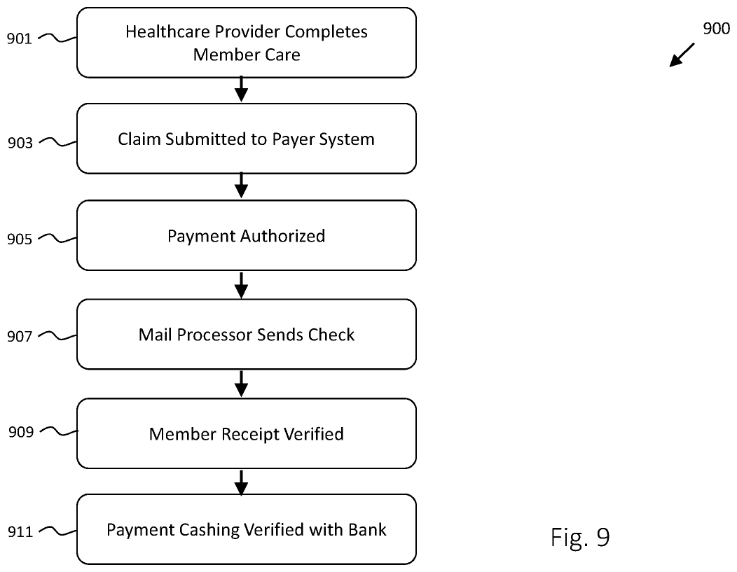

In one embodiment, an API is used by the Control/Verification Manager 809 to communicate with various Healthcare Providers, members, pay systems, and government entities e.g. Medicare or State Sponsored Health Plans). The API allows various IT systems with various formats to communicate under one platform and is used for obtaining data, including updates fee schedules, and negotiated rates, and import them into the database 811 to be recorded. A further flow of the process is described below in FIG. 9.

FIG. 9 is a flowchart of one exemplary process for ensuring timely payments and verification of payments delivered, according to the embodiments of the present invention. The flowchart utilizes the system of FIG. 8.

At 901, the Healthcare Provider 805 completes providing care to a member 803. The completion means completion of a patient visit or test or lab work. The completion can be for each single visit or test or may be for multiple visits or tests. Once the completion is done, the Control/Verification Manager 809 is identified. The Healthcare Provider, or the Control/Verification Manager 809, or both record the completion of the member care in the blockchain by writing it to a block. In many instances, multiple blocks are created, at least one by the Healthcare Provider 805 and another by the Control/Verification Manager 809 and optionally by the member 803. All the blocks would report the same completion details, such as date and time of completion and the details of the completion.

At 903, the Healthcare Provider 805 submits a claim to the Payer System 807. A timely receipt submitting of the claim by the Healthcare Provider 805 and a timely receipt of the claim by the payer system 807 is critical in efficient and proper handling and payment of the claim. As such, the date and time of the submission of the claim to the payer system as well as the date and time of the receipt of the claim by the payer system is recorded by the Healthcare Provider, payer system, and the Control/Verification Manager 809 in the blockchain. Such recording provides evidence and record of timely and proper handling of the claim within the allotted time frame.

Unlike the present invention, an older system that was used in the past utilizes paper and physical handling of a claim and in such older systems there have been reports of mishandling of claims, denials that claims were either not received at all or not received in time, and other excuses and delays to either delay payment, pay less that was is owed, or ignore and avoid the claim altogether.

The present invention verifies each step, each event and transaction, such as sending and receiving date and time of the claim as well as all other relevant details of the claim, as mentioned above, records each step into the blockchain thereby providing concrete evidence and preventing any denials, delays, or excuses by the payer systems.

The Control/Verification Manager 809 logs centrally all claims sent in a secure manner in the database as well as in the blockchain such that all relevant parties can lock down when a claim was sent, received, and the details of the claim.

At step 905, the Payer System 807 reviews the claim and authorizes or approves payment. In so doing, the Payer System 807 reviews the claim in accordance with the health plan associated with the claim. For example, some health plans require an initial deductible to be met while others may not have a deductible, some health plans require that 80% of the claim be paid up to a certain amount while others may have different percentage payouts. Some health plans require copays while others don't and the amount of copay may also vary. Government regulated or sponsored health plans may cover at 100% but may not cover all medical care, such as they may not cover elective procedures that do not rise to the level of needed medical care. Further, health plans may also cover one medical procedure at a different percentage than the other and may require the patient to contribute a portion of the payment as out-of-pocket costs.

Since medical health plans include numerous rules and complex clauses and twists that require a higher level of understanding, there has been a lot of fraud associated with improper medical coding and either no payout or lesser payout than what is rightfully required to be paid to the member. In the older system, complaints are often made about payer system and their associated medical plan provider denying payment, claiming a payment was processed and authorized when it was not, claiming that a check was mailed when it was not or modifying backend transaction records to say claim was paid on certain date and time but in reality, it was not.

The Present Invention overcome these issues by having the Control/Verification Manager 809 logs centrally all authorizations of the claims in the database as well as in the blockchain such that all relevant parties can review when the authorization or approval was made and determine whether such payout was proper and in accordance with the health plan associated with the member.

Further, the Control/Verification Manager 809 is also capable or reviewing the authorization in context and connection with the medical plan associated with the member. The Control/Verification Manager 809 reviews the medical code entered by the Healthcare Provider, reviews the payout for that medical code in the contractually agreed upon medical plan, and then examines whether the payout follows the payout promised by the health plan. In instances where a medical code needs to be converted to a medical description, the Control/Verification Manager 809 does so and then determine a match with the language in the health plan with the medical code entered by the Healthcare Provider. Further, the Control/Verification Manager 809 may also do an additional verification with the member to ensure that the medical care claimed to be provided by the Healthcare Provider was in fact the actual medical care received by the member thereby preventing any wrong medical coding or excessive medical billing that results in a higher invoice/bill to either the member or any other party or government agency that is required to do the payment.

If there are any status changes to the claim, then Control/Verification Manager 809 centrally logs all status changes to claims on date time that they occurred in a secure manner in its database as well as in the blockchain. The Control/Verification Manager 809 is also capable of tracking the percent of claims by medical specialty by age range, geography, gender or ethnic backgrounds on a per member per month (pmpm) basis to see which members did not have appropriate access to medical care. Additionally, the Control/Verification Manager 809 can also rate members for access to medical services and provide a user of the system to extract several types of statistics from the data gathered.

Since Healthcare Provider fees may change from time to time, as well as negotiated amounts between a payer system and a Healthcare Provider or a government entity (e.g., Medicare or state sponsored medical program) may also change from time to time, it is important that payer systems, claims, and all processing of the claims keep up with the most current and updated fee schedules as well as calculate interest properly for any past claims that have been underpaid. The present invention's Control/Verification Manager 809 sends queries to all relevant parties as well as government entities to frequently, or in real-time, update all the fee changes and the changes to negotiated amounts such that the claims are processed with the most updated fees and paid out in accordance. The Control/Verification Manager 809 also reviews any past claims with the fee changes and updates the claim payouts as well as any time delay interest that is associated with the delayed or lesser payout to calculate and correct the payments as needed. The Control/Verification Manager 809 also interacts with several different IT systems with various distinguished formats of various health plan providers, Healthcare Providers, and state and federal medical sponsored programs through an Application Programming Interface (API) 817 to update the fee data and negotiated rates. The API communicates with these IT systems that may have various formats are converted into one format such that data can be obtained and then transformed into a format that can be recorded in the database 811. The APIs allows downloading the fee schedules in one place and in one format. The Control/Verification Manager 809 also handles bundled payment of claims or case rates from a central location.

Once the payment authorization is made, at 907 a mail processor 813 mails out a check to the member 803. The check amount is the amount that has been authorized by the payer system previously. The amount, date, payee and payor, and other relevant details of the check as well as the mailing details are recorded into the blockchain. The Control/Verification Manager 809 can either centrally mail out checks through the mail processor or centrally cause ACH type of payments to members, Healthcare Providers, and other entities entitled to receive the payment.

In addition to mailing out checks, the mail processor 813 can also be used for other mailing functions. For example, a regulatory agency, such as Medicare can use the mailing processor 813 to send out letters to an Independent Practice Association (IPA), a specific Healthcare Provider, or other medical professional or facility. The letters may be for a wide variety of reasons, such as compliance, updates to Medicare etc. Additional types of mailings, as well as tracking of the mailings is also performed by the mailing processor 813. The tracking provides verification that a letter was sent and if a repose was received. Further, letters requiring a signature can also be digitally signed and stored by the Control/Verification Manager 809 in the database 811 as well as on the blockchain thereby providing evidence during an audit.

The Control/Verification Manager 809 also allows Healthcare Providers or members to log complaints, such as improper payment, check delay or any other issue relating to the payment of the claim.

At 909, the member posts the receipt of the check to a block in the blockchain. The posting process involved the member electronically scanning the check, taking an image of the check using their mobile phone, or transferring bank verification, and then posting that electronic file or image to a block in the blockchain.

At 911, the cashing of the check, or the wire being received in the member's bank is verified. The verification can be through requiring Payers to upload bank statements which will be processed centrally using OCR to match up with claims payment records, or have the member upload a cashing receipt, or have an API with the bank and authorized access to determine if the claim check was deposited into the member or the Healthcare Provider's bank account thereby providing tracking of the payment.

Since the details of the claim are recorded in the database and the blockchain, any disputes or resolution of claims is handled through the evidence recorded, including any changes to claim and payouts. All such disputes and appeals are logged centrally in the database by the Control/Verification Manager 809.

If providers have a dispute, they can file a Provider dispute resolution (PDR). In older system, Payers have ignored disputes and stated that they never received them. Payers have also logged disputes and claimed that they have appropriately handled or resolved them when in reality, they have done nothing but just updated backend data. The present invention can log all PDR from start to finish in a central place and in a secure manner where all relevant parties can see the PDR status at all times. If a PDR or related correspondence is not being processed in a timely manner, the present invention can provide an alert or a warning.

One example of the dispute resolution through the present invention is described below. Another example will be described in FIG. 10. In one scenario, a Healthcare Provider may have a complaint for a payment from the Payer and as such may file a PDR. The PDR would be centrally logged and all parties associated with the dispute, such as Healthcare Provider, Payer. Health Plan Provider, Member may be able to view that a dispute has been filed. Alternatively, the Control/Verification Manager 809, may provide an alert or a message through the API to all connected parties that a dispute has been filed. The details of the dispute as well as the time/day the dispute has been named would also be visible to all the parties. As the dispute resolution progresses and additional parties or the responding party (payer) provides a response, all progress, times and details of responses, and the timeline of the resolution would also be centrally logged and all relevant parties would be able to view the details. Alternatively, I addition to centrally being logged, all events and communication would also be recorded in the blockchain.

Since all the evidence and relevant data pertaining to a PDR can be found in the central database as well as in the blockchain, the Control/Verification Manager 809 is capable of automatically adjudicating the complaints, claims, and disputes raised by the parties through use of stored data. As such, the cumbersome function of an audit and gathering of evidence is eliminated by the auto adjudicating function of the Control/Verification Manager 809.

I addition to benefiting the parties in dispute resolution, the present invention also aids the parties in expeditious exchange of information, such as in real-time, that may be needed to process a claim. At times, a claim may be missing details that are required by the payer to process the claim. Certain Payers in the past have taken advantage of the missing information to delay the processing of the claim or simply not respond. The present invention logs all data centrally and alternatively in a blockchain. As such, the status of the claim, the processing timeline, along with all content of the claims are available to all the parties and optionally available in real-time. A party that needs the missing information can flag the claim as such and all the relevant parties would see which piece of information is missing and have the ability to respond or post the missing information. Alternatively, the Control/Verification Manager 809 may send a message or alert to the parties that holds the missing information such that they may expeditious respond or post the missing information thereby expediting the whole process for claim payments.

FIG. 10. is a block diagram representing one embodiment of an automated dispute resolution process, according to according to the embodiments of the present invention. The process 1000 may use either of the Systems described in FIG. 2, 3, or 4.

At 1001, a Healthcare Provider reviews a claim. All the claims are centrally logged by the Brain 310 or Control manager 410 of the system in a central database, e.g., database 450. A claim is an instrument, document, or process, such as an invoice or bill, by which a Healthcare Provider provides a list of medical codes that correspond with the care provided to a patient and submits the instrument for a Payer for getting paid for the service provided.

Since a Healthcare Provider may work on several patients, or a single patient on multiple occasions, there may be several claims that relate to each Healthcare Provider that are stored in the database. The Healthcare Provider may review all their claims periodically, at a set time after the claim has been issued, or at the end of each billing cycle, to determine if the claim has been paid. The Healthcare Provider may also access the database to pull up only those claims that have not been paid or claims that are overdue over a certain period, basically, the Healthcare Provider may use several search and screening options to review a single or multiple claim.

In this scenario, the Healthcare Provider identifies a claim has it has reviewed and identify a particular claim that has not been processed or has not been processed properly. There may be several issues with such a claim, including, no payment, partial payment, incorrect payment, overdue or some other reason due to which the claim has not been paid to the Healthcare Provider for a service they provided for a particular patient.

The Healthcare Provider at 1003 initiates a PDR. As mentioned above, the PDR is centrally logged by the Control manager 410. Additionally, the PDR is also reported to the blockchain by both the Healthcare Provider as well as the Control manager 410. Further, the Control manager 410 may also send an alert to all parties in that are in the network and authorized to receive confidential information in reference to the particular claim or the patient. In some instances, an encrypted key may be sent separately to all the relevant parties and those that desire to review the claim may decrypt the encoded key to view the message. A cryptographic algorithm may be used by the system to generate the encrypted key.

At 1005, the Control manager 410 sends the PDR to the Payer that is responsible for payment to the Healthcare Provider. The sending of the PDR is logged centrally as well as posted to the blockchain by the Control manager 410. The responsible Payer is determined by the healthcare plan associated with the claim.

At 1007, the Payer may process the claim and select to pay the claim, deny the claim, or require additional information from the Healthcare Provider to pay the claim. The decision made by the Payer is then provided to the Control manager 410 and recorded in the central database as well as posted to the blockchain.

The decision is then communicated to the Healthcare Provider at 1009. If the decision is to require further information that is missing from the Claim, the Healthcare Provider and Payer may exchange such information. The Payer's decision, regardless of what it may be, the key is that both the Payer and the Healthcare Provider is in agreement to resolve the issue. However, if the Payer's decision is unacceptable and inconsistent with the Healthcare Provider's records, that is when the automated dispute resolution takes place.

As such, at 1009, if the Healthcare Provider agrees with the Payer's decision and accepts the decision, then the Claim is closed at 1011. However, if the Healthcare Provider does not agree with the Payer's decision then the Healthcare Provider has two options. It may either resubmit the Claim or Complain to a higher authority or a separate entity. The decision is communicated to the Control manager 410 and both centrally logged as well as posted to the blockchain.

If the choice is to resubmit the claim, the process is fed back into step 1005. Steps 1007 and 1009 are then repeated until the claim is closed. Each step in the automated claim resolution process is communicated to the Control manager 410 and posted to the blockchain.

The process may allow a certain limit to resubmissions. For example, it may allow two or three resubmissions or there may be a preset limit. The process allows one or more attempts for the Payer and the Healthcare Provider to resolve the dispute, however, if the resolution is not reached, then the Healthcare Provider may complain or raise the issue higher at step 1015. Once again, the Control manager 410 both centrally logs as well as posts the complaint to the blockchain. For example, the complaint may be made to Medicare, the Health Plan provider, or some other higher or separate entity that manages the Payer or has some authority to review issues relating to the Payer and possibly can have a financial impact on the Payer for improper practices or fraud.

Once the complaint is raised and additional entities are informed and become part of the resolution process, the claim is once again sent by the Control manager 410 to the Payer for resolution at 1017. Having gotten an additional chance to resolve after the complaint, the Payer may resolve the PDR, in which case the claim is closed at 1011, or the Payer may once again deny the claim. Upon denial, steps 1015, 1017 and 1019 are repeated.

The system, through its the Control manager 410, may automate the process and then after one or more cycles, or a determines number of cycles, may pull all the record from both the centrally logged databases, blockchain, and any input provided by the separate entities and then resolve the claim in favor of either the Payer, the Healthcare Provider, or do a split decision with a certain percentage in favor of the Healthcare Provider and remaining percentage in Favor of the Payer. Optionally, the Control manager 410 may also mail a check or authorize a bank wire to the Healthcare Provider in accordance with the decision made by it. As such, the process, the resolution, and payment may be automated thereby providing a complete dispute resolution, audit, as well as settlement by the system.

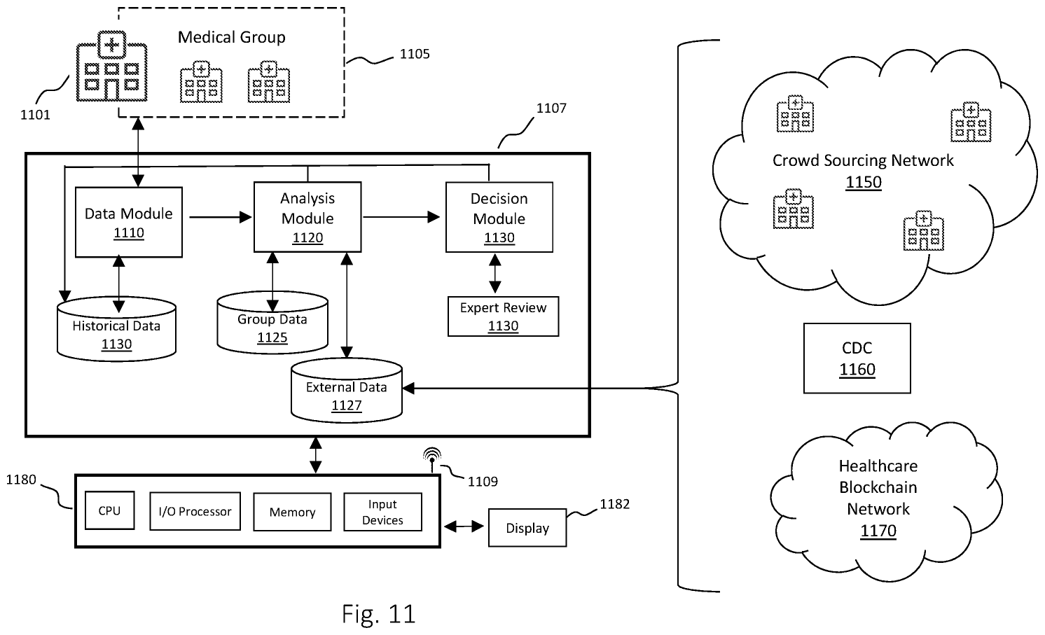

FIG. 11. is a block diagram for performing error checking and predictive analysis for generation and payment of claims, according to the embodiments of the present invention. The healthcare blockchain system, as shown in FIGS. 1-4 include modules for collecting medical and patient data, analyzing and transforming the medical and patient data, and determining through a plurality of logic tree embedded decision trees to determine if a submitted medical code, claim, or request conforms to the practice, geographical area, and patient type based on historical data, group data, and data from external sources. For example, if a clinic in Chicago reports a case of Ebola Virus, which is rare and not a commonly found disease or virus in Chicago, then the system performs analysis on historical data, obtains data from various external sources and determines whether the claim should be flagged for further review or decide that the claim was made in errors. Likewise, if a specific clinic processes claims for a certain medical condition, for example Mesothelioma is a rare disease that occurs in about 3,000 people in U.S. per year and the rate of occurrence has been declining. If a clinic processes an unusually higher number of claims relating to Mesothelioma, then the Machine Learning and Data Enhancement Module 1107 flags such claims for further review or decides if they are made in error. The goal of the Machine Learning and Data Enhancement Module 1107 is to detect and flag errors, flag unusual activity, flag activity that is historically not aligned with prior claims or practice of the clinic, flag claims that are excessive in amounts, and flag any other medical reporting or claims activity that is outside the standard and routine practice of the specific clinic or Healthcare Provider. In addition to flagging, the Machine Learning and Data Enhancement Module 1107, which includes standard computer functionality, is capable of sending out alerts or mailing to all or selected parties in its network informing them of the flagged issue.

The Machine Learning and Data Enhancement Module 1107 also function to learn over time and identify potentially fraud or incorrect claims before they make their way through the payment system. For example, if a medical practice does not have a certain medical specialty and a claim with a medical code that is associated with such specialty is being processed, then the module 1107 flags the claim as a possibly fraudulent claim. In operation, the module 1107 checks the credentialing of the medical practice, any prior claims with that specialty, and new updates, among other things, and then determines that the claim is likely an error or fraudulent. Claims that may have errors or an indication of fraud, such as claims for overbilling, billing for a doctor on vacation, billing for a doctor not in the practice, billing for medical tests not ordered or performed, billing more than once for a patient seen only once are flagged by the module as well. Although some examples of errors and attempted fraud were discussed, other examples or errors or possible fraud are also detected by the Machine Learning and Data Enhancement Module 1107 and flagged for review or an alert is provided to other entities in the system to further review the claim and act.

A clinic, or a specific medical practitioner, 1101 is displayed. The clinic 1101 may or may not be part of an extended group. For example, in one embodiment, the clinic may be owned or be part of the same medical group 1105 that operates or owns two additional clinics.

The Deep Learning or Machine Learning and Data Enhancement Module 1107 includes a Data Collection Module 1111, an Analysis Module 1120, and a Decision Module 1130. The respective modules are communicatively connected to various Data Sources, either internal or external.

The Machine Learning and Data Enhancement Module 1107 is part of a computing system 1180 which includes a CPU, memory storage module, I/O Processor module, Input devices, and a standard bus, an antenna 1109, and a wired or Wifi network for communicating, receiving, and sending messages to all required parties. It is also coupled with a Display 1182 for displaying the claim or any part of the processing stages.

As shown in FIG. 11, the various modules (Data, Analysis, and Decision) store data in various data structures and databases to keep track of the healthcare related information sources, caregiver (e.g., physician, specialist, clinic, hospital etc.) requests or claims, queries, and answers involved in the healthcare administration and management process.

The Data Module 1111 is communicatively coupled to the Historical Data sources database 1130. This database includes all the historical data of that specific clinic 1101 and if the clinic is part of a larger practice, then it may also store historical data of other clinics and Healthcare Providers in its medical group 1105. The historical data may include past patient data, type of claims, amounts of claims, types of medical codes previously prescribed, and types of medical diagnosis and tests ordered or performed. It may also keep historical data on past referrals from the clinic or medical group, past claim and their associated amounts, frequency and number of claims for each type of medical code, data on type of patients (e.g. age, ethnicity, medical history) and other relevant data that is relevant to administering care to their patient and processing claims.

Although a variety of information is stored in the Historical Data Database 1130 and the functionality of the Machine Learning and Data Enhancement Module 1107 can be applied to any of the data relating to medical care or processing, for exemplary purposes we focus here on medical claims processing.

As such, in this embodiment, the Historical Data sources database 1130 is used by the Data Module 1111 to obtain data relevant to a type of claim that is being processed by the Clinic 1101. The Data Module 1111 selects, from among multiple historical information sources in the historical information source database, one or more suitable information sources to provide useful information for the comprehension and fulfillment of a user request to process a certain healthcare claim. In some embodiments, the historical database or the Data Module's use is optional, and a criterion or limit may be programmed into the Historical Data sources database 1130 such that if a certain type of claim exceeds the limit then it gets flagged.

Historical Data sources database 1130 may also generate one or more queries for each healthcare claim processing request for which historical information to be obtained and a data analysis is to be performed. The queries are generated based on the user request and its context information. The query is designed such that they are likely to bring back answers helpful in the comprehension and fulfillment of the clinic's claim processing and payment request. For example, if the Clinic 1101 is submitting a Medicare Claim for reimbursement for a patient to get a Magnetic Resonance Imaging (MRI) test done due to a possible brain tumor for a patient, then historical data for all MRIs as well as historical information for all neurology practices that provide care for tumor related care may be pulled from the historical database 1130.

Once the data is obtained, the Data Module 1111 further parses through the data and sends relevant data to the Analysis Module 1120 for further processing. The Analysis Module 1120 is used by the Machine Learning and Data Enhancement Module 1107 to analyze the requested claim and ensure that the claim is valid, error free, and falls in line with historical data of the clinic and also falls in line with other clinics in the same geography or specialty and does not raise any red flags—but if does not pass or meet the programmed rules or limits, then it is flagged for higher review.

The Analysis Module 1120 is communicatively coupled to a Group Data Database 1125 as well as External Data Database 1127. As mentioned earlier, if the Clinic 1101 may be part of a Medical Group 1105 and if so, then the data is typically shared under the same umbrella between the clinics or medical practitioners in that medical group. The Analysis Module 1120, through its Group Data Database 1125 can query and obtain relevant data for processing the claim submitted by the clinic. In addition to historical data from other patients seen by all the clinics in the medical group 1105, the Analysis Module 1120 may also pull other data for the same patient if the patient had visited multiple clinics in the group and seen other medical Healthcare Providers. For example, if an MRI was performed for the Patient earlier by another Healthcare Provider then that information is obtained and analyzed.

Analysis Module 1120 is also communicatively connected to the External Data Database 1127 through which it sends queries to and obtains answers. The External Data Database 1127 obtains External data from various sources, including, Crowd Sourcing Network 1150, Center for Disease Control (CDC) 1160 (or other government or research institutions that report health care alerts, epidemics, or diseases in the geographical area), and Healthcare Blockchain network 1170.

In one embodiment, a designed query is crafted by the Analysis Module 1120 to obtain external data relevant to the claim that is being processed. For example, if a claim relating to Mesothelioma, or a rare heart condition that is typically uncommon, is being processed, then the query is designed to gather specific data for those diseases such that the current claim can be compared and analyzed with having additional relevant data from other third parties available. The points of comparison may be, how many other Healthcare Providers have processed claims for the same or similar medical code or disease, the time frames associated with all the claims, the costs and payments requested for such claims, any reporting of higher number in the geographical area or by age, gender, or ethnic group of the disease to analyze any outbreak, and other relevant data to compare whether the claim being processed fits in with others or is an outlier.

The answers to the queries from information sources are sent to the external data database 1127 by external sources. For different pieces of information sources and/or queries, the time frame in which monitoring for answers is performed can be preselected. The external data database stores the answers received for each query in the database and keeps track of the answer statuses of the queries.

Once the Analysis Module determines that sufficient answers have been collected for the queries issued for a particular user request, the answer integration module filters, ranks, reconciles, and integrates the answers to provide consolidated sourced information relevant to the particular user request and analysis the data. If the responses are unsatisfactory, then a second or third redesigned query may be sent and the process repeated.

In this instance, when faced with a claim for Mesothelioma, the number of Mesothelioma cases reported can be obtained from the Crowd Sourcing Network 1150, CDC 1160, and blockchain Network 1170. If the clinic is part of a larger network thereby authorizing it to obtain patient confidential data, the all the other nodes or clinics on the blockchain and their reporting may be obtained for analysis.

The analyzed data is then transferred to the Decision Module 1130 for further processing. The Decision Module 1130 reviews the analyzed data and makes a determination. The criteria for the determination may vary, for example if other clinics have reported an average of 5 diabetes cases per month and Clinic 1101 has reported 6 or 7, then the Decision Module 1130 would calculate the percentage deviation from the data set obtained and decide whether to flag or alert the parties of the claim. In some instances, the percentage deviation may be programmed into the system. For example, a 20% increase from the data set might be allowed and anything that exceeds would be flagged for review.

In one embodiment, the decision may be internally reviewed by an Expert 1130. This may be the clinic management, Healthcare Provider, or an individual in accounting or a hired consultant or third-party company. The expert review would provide an opportunity to review the claim in an electronic device and then use an application to correct, reject, or flag for further review. The Expert Review step would allow the clinics to internally handle the claim before the system flags it for review to other parties.

The data from the Analysis Module 1120 and the Decision Module 1130, as well as all the data obtained from the Group or External Sources is transferred to the Historical Data database 1130. The machine learning aspect of the present invention allows the data gathered over time to be used to enhance the processing and understanding of future related claims.

FIG. 12 is a flowchart of one exemplary interaction between the Healthcare Provider and the payer system for processing a payment, according to the embodiments of the present invention.

At 1201, the processor of the system, such as systems shown in FIG. 2, 3, 4, or 8, causes the system to generate upload a bill, the CMS-1500 bill, that has been generated by the Healthcare Provider's office. The CMS 1500 bill, also referred to as the CMS-1500, is a claim form that allows the Healthcare Provider to bill Medicare. Typically, Healthcare Providers and vendors use this form transmit health care claims electronically.

At 1203, the system checks to determine if there is an electronic copy of the CMS-1500 form. If there is an electronic copy, then at 1205, the processor of the system, causes the system to send the electronic claims data to configured clearing houses. These clearing houses act as intermediaries for processing and clearing the claims or invoices and in this case the CMS-1500.

At 1207, the clearing house validates the data provided on the CMS-1500. This includes the clearing houses to execute the claims based on regulatory or HIPAA rules. Function to exchange monies, clear trades, collect and maintain margin monies, provide error correction, and report data. The error correction may be to check dates, amounts, names of parties, proper payment in accordance with the health plan, medical codes etc. They basically provide a secondary check.

At 1209, once validation is complete, the electronic file is sent to the Payer through a secure FTP site at which point, the Payer system at 1211 stores the information in form of an 837 file in its local storage and at 1221 loads the file to the database for claim processing and payment. As an alternative, in one embodiment, the system can also prove to be synonymous to a highly secure and trustable DATA EXCHANGE eliminating the need for external communication mediums like FTP, Mail, Faxes, and EDI.

At 1203, if it was determined that an electronic copy of the CMS-1500 form is not available in the system, which implies that only a paper copy is available, then at 1213, the paper claim is sent directly for the Payer's office and it is scanned and sent back to clearing house as an electronic file at 1215.

At 1217, the clearing house validates the claim, similar to the validation at 1207, and then generates an 837 electronic form. This form is used to confirm to HIPAA requirements for electronic submission of healthcare claims. It includes, among other things, amounts of payment for care provide to a patient.

At 1219, the generated 837 electronic form is loaded in the system and then sent to the Payer through the secure FTP site. Steps 1211 and 1221 follow after the electronic data is sent for it to be saved locally by the Payer and then processed and paid.

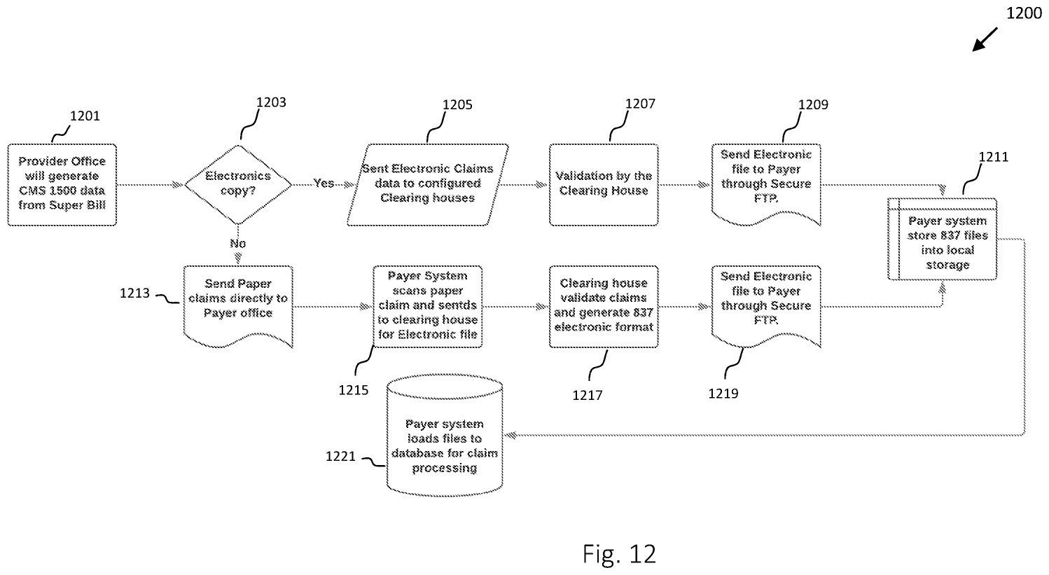

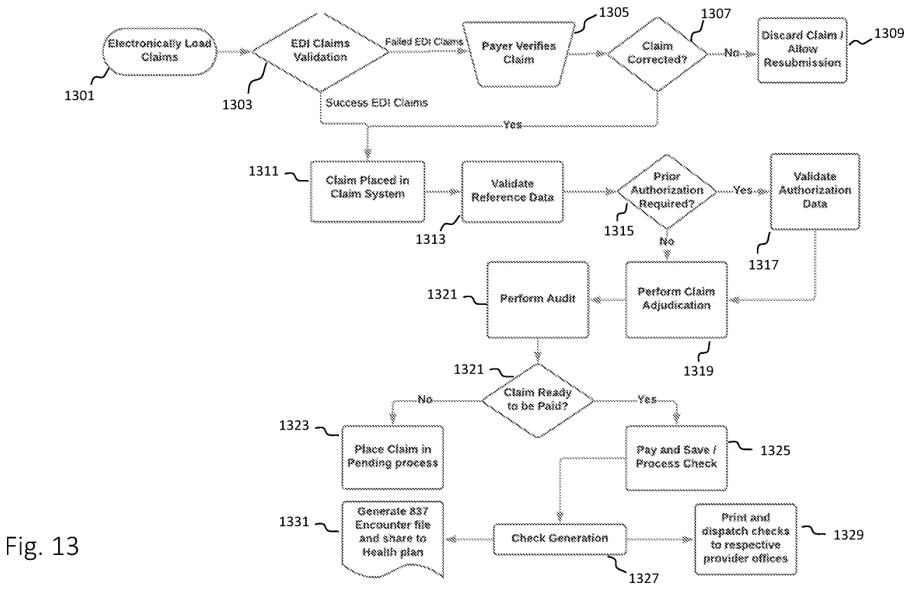

FIG. 13 is a flowchart of one exemplary process used by the payer system to validate a claim and provide claim adjudication, according to the embodiments of the present invention.

At 1301, the processor of the system, such as systems shown in FIG. 2, 3, 4, or 8, causes the system to load electronic claims into the Payer's Database. This database may be same as the central database or a separate database that is at the Payer's site. Alternatively, the claims can be loaded manually to the system as well.

At 1303, the loaded claims, which are Electronic Data Interchange (EDI) Claims, are validated by the processor. The validation process includes error checking of the claim. If the validation is failed, then at 1305, the Payer System or Payer's staff electronically verifies the claims. The verification may include information to correct any errors in the claim.

At 1307, the system checks to determine if the claim correction provided by the Payer was used to correct the Claim. The processor electronically performs this check by using the Payer correction data and then reevaluating the Claim to determine if the Payer data is now included in the revised claim.

At 1309, if the system determines that the Payer corrections were not incorporated and the Claim was neither cleaned nor corrected, then the system discards the claim and provides a notification to resubmit the claim.

At 1303, if the validation was passed, i.e., it was determined for the Claim to be valid. Then at 1311, the claim is placed in the claim system and then at 1313 the claim's reference data is validated by the processor. The reference validation includes checking if the parties, such as member or Healthcare provider, are in good standing and if their information is correct and updated.

At 1315, the system determines if a prior authorization is required. In certain healthcare plans, including certain Medicare of regulatory healthcare plans, there are several instances where a prior authorization is required before the next step is taken. For example, in some cases a prior authorization is required to see a specialist, or a prior authorization is needed to process a payment, or a prior authorization is needed for a certain type of bill or payment.

At 1317, if a Prior Authorization is required, then the system validates the authorization data. This means the system checks to determine if in fact an authorization was approved and properly communicated prior to the next step.

At 1319, if a claim prior authorization is not required, as it may not be required for certain medical procedures, for certain payments, or for smaller amount payments, then at 1319 the system performs a claim adjudication. This may also be performed after step 1317. The claim adjudication includes performing any error correction, reaching a resolution between two parties when there is a dispute or errors in the claim.

At 1321, an Audit is performed. The Audit may include testing the claim against a plurality of criterion. For example, it may be tested for dates of service, payment amount, medical codes, type of service rendered, payment account and contact or mailing information.

At 1323, the system determines if the claim is ready to be paid. If the answer if NO, then at 1323, the claim is placed on hold until the errors or issues are resolved.

At 1325, If the answer if YES, then at 1327, the claim is processed, the information is saved and the payment is made by either processing a check and having the mail processor 813 mail out the check or have the funds electronically transferred to the recipient's account.

At 1327, a check is generated in the amount of the claim and at step 1329 the check is printed and dispatched to the provider office and at Step 1331 an 837 Form is filled and the file is shared with the health plan provider.

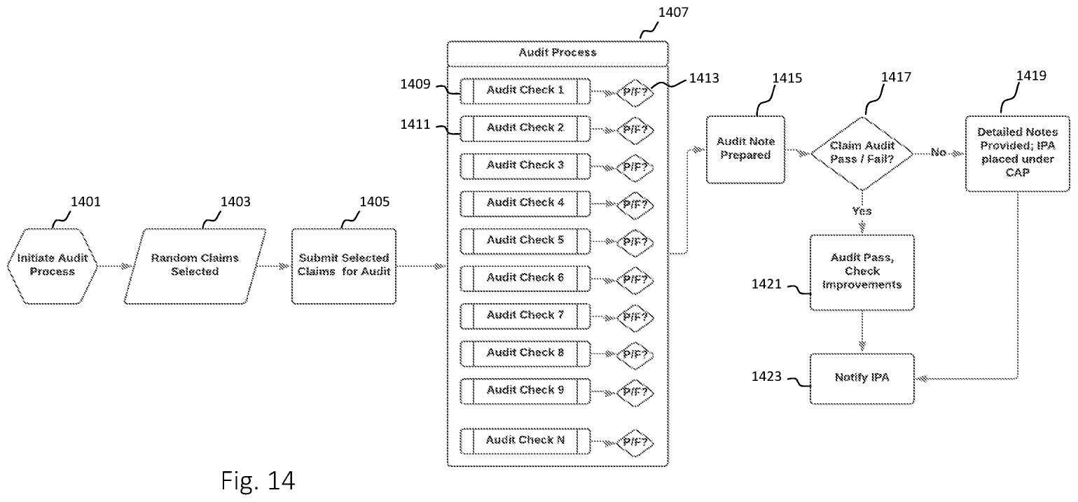

FIG. 14 is a block diagram of an automated audit process, according to the embodiments of the present invention. The automated audit process reviews transactions for a certain time period and selects a number of sample transactions from the entire data set for analysis. The selected sample is then analyzed against a plurality of criterion.

At 1401, an audit process is initiated. The system prepares the claims for audit by screening claims based on a criterion and organizing them. The screening criteria include screening by dates, quarters, a particular month, a certain Healthcare Provider, the entire practice, for a specific patient, or for a specific clinic or group. The criterion may also be determined based on a regulatory agency requirement of the type of data set to be audited.

In one exemplary embodiment, claims for the month of January, February, and March, i.e., the first quarter of a year, are selected for a specific Healthcare Provider. As such, the system would review all the claims for the provider, select the claims for January, then select the claims for February, and then select the Claims for March. The claims would be screened to ensure that they all relate to one specific Healthcare Provider. Alternatively, the claims can further be narrowed for a single specific provider and a single specific payer system or regulatory agency. For example, of the purpose of the audit is to ensure that all claims relating to Medicare have been processed corrected, then the screening criteria would be narrowed to claims for January-March for the specific Healthcare Provider that relate only to Medicare.

At 1403, a random set of claims from the entire data set screened and narrowed in step 1401 are selected. The random set is a sample size of the entire data set narrowed in 1401. The sample size maybe predefined to a certain percentage or the sample size may be a certain percentage or number that is a minimum requirement from the regulatory agency. The sample size may also provide additional criterion and split such as a certain number of claims from January and a certain number of claims from February and March to get a date spread sample size. The sample size may also be by selecting two different physicians in the same practice.

At 1405, the sample selected is submitted for audit. This includes running an executable program that is stored in a database, such as database 450 in FIG. 4 or database 811 in FIG. 8 or memory module 1180 in FIG. 11. Although a sample set has been selected, the information forwarded for audit would also include a summary of the entire data set, such as the total number of claims for January or the entire first quarter, average amounts charged per claim, high and low amounts, or other desired summary data that would provide a snapshot of the entire data set.

An audit process 1407 is then executed and applied to the sample data set from step 1403. The audit process 1407 may include several audit criteria that are preselected, selected based on a requirement of a regulatory agency, selected based on historic audits, selected based on best practices in the field, or user defined.

For example, one audit criteria may be Audit Check 1 (1409) for auditing the total number of claims for January, or the entire first quarter for Medicare claims. In this example, the system would either access the Department of Health Care Services (DHCS) records or use a number provided by the DHMC on number of claims that were processed during January or the entire first quarter.

As such, the system would compare the number of claims from the database for January and try to match those numbers with the number or claims received for January from the DHCS, basically compare the internal or central database numbers with the database numbers for the Medicare or DHCS database.

The processor, such as processor 1180 in FIG. 11, Analytics Engine 250 or Deep Learning Engine 270 in FIG. 2, then processes the data sets and the numbers from the DHCS to determine a pass and If the numbers match, the processor determines a Pass at the Pass/Fail (P/F) gate 1413 and reported. Alternatively, if a match is not made, then the processor would conclude a Fail at the Pass/Fail (P/F) gate 1413 and report the Fail. The system may also do a re-determination based on any notes provide in the central database or the blockchain.

Yet another example of an audit criterion may be Audit Check 2 (1411). For example, Audit Check 2 (1411) is to determine if the bank statements for the Healthcare Provider match the amounts claimed to be paid by the Payer.

In this example, each claim represents a medical service provided by the specific Healthcare Provider. Each claim also represents payment owed to the Healthcare Provider for the service provided on that claim that needed to be paid by the Payer. In response to each claim, the Payer would have paid the Healthcare Provider for the amount invoice or billed (through a super bill or otherwise) and sent to the Payer for payment. The Payer would then pay the amount listed on the claim or engage in an information exchange or an automated dispute resolution for that specific claim and then pay the amount listed on the claim. In any case, for explanatory purposes, the amount invoice, the amount paid, and the amount received by the Healthcare Provider needs to match since its essentially the same amount. In some instances, the amount may be different but a valid explanation may be provided that was acceptable to both Healthcare Provider and payer. All the transactions mentioned here would be recorded in a central database as well as posted to a blockchain as explained earlier in FIGS. 1-13 thereby providing all the details of the transaction that are authenticated and tamper proof.

Audit Check 2 (1411) is processed using a processor, which then processes the sample data set from Step 1403. Each claim is the audited for payments and bank verification. This process may include, the system obtaining the amounts invoiced on each claim for the month of January. Then the system may obtain data from the Payer for each claim to determine the amount paid, or claimed to be paid, by the payer.

Once these sets of data are obtained, then the system would a) either access the Healthcare Provider's bank account based on access authorization provided by the Healthcare Provider or b) require the Healthcare Provider to upload bank statements into the system, such as through an API 270, 324, or 817. The processor of the system would then cause the system would then check amount received by the Healthcare Provider on each of the claims processed.

The processor of the system would then cause the system to determine a match between the amount received on a specific claim by the Healthcare Provider with the amount paid or claim to have been paid by the Payer on that specific claim. The first step would be to determine a match. If a match is determined, it would imply that both parties are in agreement with the amount paid and the amount received, and the processor would conclude by determining a Pass at the Pass/Fail (P/F) gate 1413 and report the Pass.

If a match is not made, then the processor would conclude a Fail at the Pass/Fail (P/F) gate 1413 and report the Fail. It may also be the case that there is a valid reason for the numbers not to match. For example, if an error had occurred in the original bill from the Healthcare Provider to the Payer, and the Payer had requested a correction or some other affirmation of the incorrect amount from the Healthcare Provider, and both parties had agreed to the changed amount; notes of the conversation and transaction which would be logged both in the central database as well as in the blockchain; then the system would look for additional verification, explanations, and notes in both the central database and the blockchain as a second pass on a Failed audit for Audit Check 1411. The processor would then engage an Artificial Intelligence or Deep Learning Engine, e.g. 260 or 310, analyze the notes and transactions in context of the failed claim and determine whether the explanation is valid. For example, the central database and blockchain would keep a record that verifies the notes. It would be very hard to change the records and as such they would be nearly tamper proof.

In one embodiment, a match is not made, yet an explanation provided in the note is considered to be valid thereby allowing the system to Pass the audit. This may occur when the notes reflect a more recent understanding or resolution between the parties, especially since the notes may have been entered after the invoice. In other words, the notes may have ratified or changed the invoice. In such a scenario, on the second pass, the system would redetermine the audit criteria and render a Pass. Other alternatives, such as mismatch of notes with actual payment may render a Fail. In any case, the system has the capability of running through one or more passes on a Pass or Failed data claim which would include providing a feedback into the process and re-starting the audit process 1407 now with additional data obtained from the central database and the blockchain.

Further, in the event of a second pass, for example for cases in which the notes and transaction data is verified, however the invoice data is not matched, then in another alternative embodiment, the system may also notify the parties involved and wait for response to complete the audit. Alternatively, the system may conclude that Audit Check N is not complete and awaiting information.

Although just two examples of Audit Checks were detailed above, a plurality of additional audit checks, such as Audit Checks 3-9 and N more Audit checks may be performed on the sample data set. These may include audit for medical codes, audit for an amount allowed per claim by a regulatory agency and the actual amount invoiced and paid, audit for any underpayments, overpayments, audits for any corrections, audit for interest payments on delayed claims.

Additionally, the system can also track and data changes or tampering of the data on any of the claims. For example, if a claim was paid on January 2^{nd} and the Payer tried to backdate the claim to December 31^{st}, the close of the prior fiscal year, the audit system has the capability of detecting changes made to the data and tracking and reporting such change.

At 1415, The processor of the system would then cause the system to prepare an electronic audit note. This audit note would include a summary of the audit, such as types of criterion tested, results of each test, number of Passes and Fails, attempts at second passes, additional notes added or obtained from central database and blockchain, any data changes or attempts made to change or tamper the data, and other desired results for an audit note that is either desired by the user or required to be on an audit note by a regulatory agency or audit note based on industry standard practices.

At 1417, The processor of the system would then cause the system to Pass or fail the entire audit 1407. This would include a Pass/Fail based on all the Audit Checks 1-N, not just one of the audit checks. The Pass/Fail may be determined based on several options. For example, one option would Pass the entire Audit only if all the Audit Checks 1-N are Passed, another option would be to Pass if a certain percentage of the Audits in the Audit Checks 1-N are Passed, yet another option would be to Pass only if all Audits specified as critical or of a high importance are Passed. Other overall Audit Pass/Fail criterion may be specified by the user or a regulatory agency or be based on best practices. In one instance the system would Pass the Audit and then proceed to Step 1421.

At 1421, the processor of the system would then cause the system to suggest improvements. Since the system interacts with deep learning engines and modules 260 and 310, where there is a constant improvement of the system, predictability, and claim enhancement based on various ongoing data inputs, crowdsourcing, and historical data, along with data from industry practices, industry updates, and regulatory updates, the system is capable of making suggestions for improving the claim processing and audit process' efficiency and accuracy. As such, at 1421, the system may suggest improvements to either the Healthcare Provider, Payer, for future enhancements or to come up to speed to best practices or to reduce errors.

At 1423, the system informs the Independent Practice Association (IPA), i.e, the Healthcare Provider or Specialist. The information may include suggestions for future audits, best practices, regulatory information etc.

At 1417, if the Audit is Failed, then the system at 1419 would provide detailed notes of the audit and details of the failure(s) to the IPA. If the failure is severe, or if the failure is beyond allowable threshold that may be set by the user, the auditor, or the regulatory agency, then the IPA is placed under a corrective action plan (CAP). The IPA may also be placed under a CAP if they have failed a similar audit in the past and the systems detect repeat occurrences. Another criterion for placing the IPA on CAP may also be used.

FIG. 15 is a block diagram of a computer system that can is used in operation of the centrally storing healthcare transactions, providing automated dispute resolution, performing predictive analysis on claims, performing an audit, automatically verifying Healthcare Providers and their updated fees, posting healthcare transactions to blockchain and other features disclosed in the present invention, according to some embodiments.

The embodiments described herein, including systems, methods/processes, and/or apparatuses, may be implemented using well known servers/computers, such as computer 1500 shown in FIG. 15. For instance, transactions, communications, events, and functionality as described in FIGS. 1-14 can be implemented using the computer system of FIG. 15.

Computer 1500 can be any commercially available and well-known computer capable of performing the functions described herein, such as computers available from International Business Machines, Apple, Sun, HP, Dell, Cray, etc. Computer 1500 may be any type of computer, including a desktop computer, a server, tablet PC, laptop, or mobile communication device, etc.

As shown in FIG. 15, computer 1500 includes one or more processors (e.g., central processing units (CPUs)), such as processor 1504, processor 813 from FIG. 8, or processor 1180 in FIG. 11. Processor 1504 may additional modules as described in FIGS. 1-14. Processor 1504 is connected to a communication infrastructure 1502, such as a communication bus. In some embodiments, processor 1504 can simultaneously operate multiple computing threads.

Computer 1500 also includes a primary or main memory 1506, such as a random-access memory (RAM), or database 450, database 811. Main memory has stored therein control logic 1528A (computer software), and data.

Computer 1500 also includes one or more secondary storage devices 1510. Secondary storage devices 1510 include, for example, a hard disk drive 1512, a Solid-State Drive, NAND Flash Storage Drives, 3D XPoint Drives, and/or a removable storage device or drive 1514, as well as other types of storage devices, such as memory cards and memory sticks. For instance, computer 1500 may include an industry standard interface, such as a universal serial bus (USB) interface for interfacing with devices such as a memory stick.

Removable storage drive 1514 interacts with a removable storage unit 1516. Removable storage unit 1516 includes a computer useable or readable storage medium 1524 having stored therein computer software 1528B (control logic) and/or data. Removable storage drive 1514 reads from and/or writes to removable storage unit 1516 in a well-known manner.

Computer 1500 also includes input/output/display devices 1522, such as monitors, keyboards, pointing devices, etc. Computer 1500 further includes a communication or network interface 1520. Communication interface 1518 enables computer 1500 to communicate with tablet, laptops and mobile devices. For example, communication interface 1518 allows computer 1500 to communicate over communication networks or mediums 1522 (representing a form of a computer useable or readable medium), such as local area networks (LANs), wide area networks (WANs), the Internet, with Healthcare Providers, Payer Systems, Regulatory Agencies, and Blockchain network. Network interface 1520 may interface with remote sites or networks by using wired or wireless connections. Examples of communication interface 1518 include but are not limited to a modem, a network interface card (e.g., an Ethernet card), a communication port, a Personal Computer Memory Card International Association (PCMCIA) card, etc.

Control logic 1528C may be transmitted to and from computer 1500 by using the communication medium 1542. Any apparatus or manufacture comprising a computer useable or readable medium having control logic (software) stored therein is referred to herein as a computer program product or program storage device. This includes, but is not limited to, computer 1500, main memory 1506, secondary storage devices 1510, and removable storage unit 1516. Such computer program products, having control logic stored therein that, when executed by one or more data processing devices, because such data processing devices to operate as described herein, represent embodiments of the invention. For example, Control logic 1528C may be used to perform an audit, provide dispute resolution, parse through claims, verify a claim, authorize a payment, post to a blockchain, retrieve selected data from a central database, obtain crowdsource data or submit claims to a clearing house. Additionally, the control logic 1528C may be used in any of the flowcharts or operations, such as those represented in FIGS. 1-14, that require analysis or decision-making.

Although the present invention has been described in connection with several embodiments, the invention is not intended to be limited to the specific forms set forth herein. On the contrary, it is intended to cover such alternatives, modifications, and equivalents as can be reasonably included within the scope of the invention as defined by the appended claims.

What is claimed is:

1. A computerized method of recording, verifying, and auditing a healthcare transaction, wherein the transaction is related to providing healthcare for a patient that has a medical health plan, comprising:
providing a centralized control manager for obtaining, storing, and analyzing healthcare transaction data, wherein the centralized control manager stores data related to a healthcare transaction in a central database;
operating the centralized control manager through a processor, wherein the processor executes instructions stored in the central database and causes the centralized control manger to perform its functions;
electronically connecting the centralized control manager to at least two entities, wherein the first and the second entity bi-directionally communicate with the centralized control manager and each other using an electronic device over a computerized network;
the first entity executing a first electronic healthcare related transaction, wherein the healthcare transaction is digitized into a header and a body, wherein the header includes date and time of the transaction and the entities involved in the transaction and the body contains the content of the transaction;
the processor causing the centralized control manager to retrieve the data of the first healthcare transaction executed by the first entity and recording the transaction in the central database;
analyzing the first transaction and determining a standard deviation from historical and crowdsourcing data for transactions that relate to similar medical diagnosis;
flagging the transaction and transmitting an alert to at least one electronic device that is used by the first entity if the standard deviation exceeds the limits previously set into the central database;
the second entity executing a second electronic healthcare related transaction, wherein the second healthcare transaction is digitized into a header and a body, wherein the header includes date and time of the transaction and the entities involved in the transaction and the body contains the content of the transaction, wherein the second transaction executed by the second entity is in response to the first transaction executed by the first entity;
the processor causing the centralized control manager to retrieve the data of the second healthcare transaction executed by the second entity and recording the transaction in the central database; and
replicating the first and the second transaction by posting the transactions to a node in a blockchain network, such that both the central database and the blockchain network can be used to verify and authenticate the transactions between the first and the second entity.

2. The computerized method of claim 1, wherein historical data is data of past healthcare transactions performed by the first entity.

3. The computerized method of claim 1, wherein crowdsourced data is data for same or similar healthcare transactions performed by other entities that are in the computerized network.

4. The computerized method of claim 1, further comprising an Application Programming Interface (API), wherein the API is downloadable on the electronic devices of the first and the second entity and provide a means for bi-directional communication between each other and the centralized control manager.

5. The computerized method of claim 1, wherein flagging the transaction further comprising electronically sending details of the transaction to a regulatory agency selected from the group of Medicare & Medicaid Services (CMS), Department of Managed Health Care (DMHC), Department of Health Care Services (DHCS), Department of Health and Human Services (HHS), and Department of Health and Human Services Office for Civil Rights (OCR).

6. The computerized method of claim 1, wherein the first entity is a medical care provider selected from the group consisting of a primary physician, a doctor, a specialist, nurse, physician's assistant, clinic, laboratory, a medical facility, urgent care, hospice care, hospital, nursing home, rehabilitation center, a medical or laboratory technician or institution, pharmacy, home health care and related professionals, dentists, and other individuals or entities in the business and profession of providing medical care.

7. The computerized method of claim 1, wherein the second entity is selected from the group of Payer, Payer System, or Health Plan Provider.

8. A computer implemented method for automated claim adjudication and dispute resolution between two parties in a healthcare environment, wherein the claim adjudication and dispute resolution relate to medical care provided for a patient having a medical health plan, comprising:
using a system comprising a server computer and a computing device,
wherein the server computer comprises a central database and a processor, wherein a server application is stored in the central database, wherein the server computer is communicatively coupled to a plurality of parties,
wherein the computing device is a server, laptop, desktop, tablet PC or a mobile device that comprises a client processor and a client database, wherein a client application is stored in the client database;
communicatively connecting the server computer to the computing device of a first party through a computer network, wherein once connected, the server application and the client application communicate with each other over the computer network;
the first party executing a healthcare transaction, wherein the transaction relates to a medical service provided to a patient having a medical plan;
the first party electronically posting the data of the healthcare transaction to a block in the blockchain;
the server computer, through its server application, communicating with the computing device of the first party, wherein communicating includes interacting with the client application to obtain the data related to the healthcare transaction, wherein the data includes the date and time of the transaction and details of the medical service provided to a patient;
the server computer electronically storing the data of the healthcare transaction in the central database and providing access to the healthcare transaction to a plurality of authorized parties that are in the computer network;
the first party electronically filing a dispute, wherein the dispute is between the first party and the second party and relates to the medical service provided to a patient by the first party;
the server computer electronically sending an alert or a message to the first party, the second party, and other authorized parties that are part of the computer network, wherein the alert or message identifies the dispute and its contents;
parsing data from the central database and retrieving data relevant to the disputed healthcare transaction;
parsing data from the blockchain and retrieving data relevant to the disputed healthcare transaction;
sourcing a response to the filed dispute from the second party;
sourcing a response from other authorized parties that are in the computer network and are relevant to the disputed healthcare transaction;
using the data obtained from central database, blockchain, response from the second party and the authorized parties as evidence to resolve the disputed healthcare transaction; and
posting the decision of the dispute resolution to a block in the blockchain and centrally storing it in the central database.

9. The computer implemented method of claim 8, further comprising, providing an encrypted key to an authorized party, wherein the healthcare transaction is only accessible by the authorized party upon use of the provided encrypted key.

10. The computer implemented method of claim 8, wherein, the authorized parties include a health plan provider, a payer, a payer system, a patient or a member of the health plan, a care provider, and a regulatory agency.

11. The computer implemented method of claim 8, wherein data relevant to the disputed healthcare transaction includes all historical transactions performed by any of the parties in the computer network specific to the disputed healthcare transaction.

12. The computer implemented method of claim 8, further comprising reporting the disputed transaction to a regulatory agency.

13. The computer implemented method of claim 8, further comprising the sever computer deciding the outcome of the dispute, and electronically transferring money from the second party to the first party.

14. A system for auditing and providing automated dispute resolution in a healthcare environment, wherein the auditing and automated dispute resolution are provided for a medical payment claim that is related to a medical service provided to a patient having a medical health plan, the system comprising:
a central unit having a processor and a non-transitory computer-readable storage medium storing a program to be executed by the processor;
the central unit having a controls manager that is operable by the central processor,
the central unit having a central database communicatively coupled to the processor and the controls manager,
a first electronic device used by a Care Provider that is communicatively coupled over a computer network with the central unit, wherein the first electronic device includes its own processor;
a second electronic device user by a Payer that is communicatively coupled over a computer network with the central unit and the first electronic device, wherein the first electronic device includes its own processor, wherein the processor of the central unit causes the execution of the program that results in the control manager
the processor of the central unit causing to obtain data for a healthcare transaction performed by the Care Provider using the first electronic device,
the processor of the central unit causing to store the healthcare transaction in the central database, and
the processor of the central unit causing to post the healthcare transaction to a blockchain; and
an automated dispute resolution manager communicatively coupled to the control manager, wherein the processor causes the automated dispute resolution manager to audit the healthcare transaction and automatically adjudicate a disputed claim in real-time between the Care Provider using the first electronic device and the Payer using the second electronic device,
wherein the auditing comprises retrieving the data of the disputed healthcare transaction from the central database and the blockchain,
wherein the automated dispute resolution comprising the dispute resolution manager to
provide an electronic platform for the Care Provider and the Payer such that the care Provider through their electronic device and the Payer through their electronic device can select options to adjudicate the disputed claim, wherein the adjudicating options include agreement or disagreement between the Care Provider and Payer or complaint to a third party,
upon disagreement between the Care Provider and Payer, the automated dispute resolution manager analyses the retrieved data, reaches a decision, and transfers a payment from the Payer to the Care Provider and closes the dispute.

15. The system of claim 14, wherein payment from the Payer to the Care Provider is performed in real-time and consist of funds transferred to the Care Provider's centralized or bank account.

16. The system of claim 14, wherein auditing further comprises:
the processor of the central unit randomly selecting a plurality of claims from a group of claims, wherein each claim relates to the same Payer, wherein each claim relates to a separate medical service provided by the care provider to a patient;
electronically submitting selected claims for an audit;
testing each selected claim against one or more criterion and posting a pass/fail result for each such condition; and
electronically preparing an audit summary.

17. The system of claim 16, wherein the criterion tested is the number of claims within a certain time period, wherein the criteria is evaluated by extracting data from the Payer to determine the number of claims processed within a certain time period, obtaining the number of claims recorded a third party for the same Payer, posting a Pass if the claims reported by the Payer matches the claims recorded by the third-party entity.

18. The system of claim 16, wherein the third party is a government regulatory agency selected from the group of Medicare & Medicaid Services (CMS), Department of Managed Health Care (DMHC), Department of Health Care Services (DHCS), Department of Health and Human Services (HHS), and Department of Health and Human Services Office for Civil Rights (OCR).

19. The system of claim 16, further comprising electronically placing the Payer on a Corrective Action Plan if the Payer fails one or more criterion.

20. The system of claim 16, further comprising electronically figuring out errors and gaps in the Payer's selected claims and suggesting an improvement to avoid similar errors and gaps in a later audit.
